SEQUENCE LISTING (30 sequences)

SEQ ID NO: 1 - DNA, 19 bp, Artificial Sequence
  Description of Artificial Sequence Synthetic oligonucleotide
agaatcagtagaggaaagt

SEQ ID NO: 2 - DNA, 19 bp, Artificial Sequence
  Description of Artificial Sequence Synthetic oligonucleotide
cggcattccgtcaggcaaa

SEQ ID NO: 3 - DNA, 13 bp, Artificial Sequence
  Description of Artificial Sequence Synthetic oligonucleotide
cggcattccgtca

SEQ ID NO: 4 - DNA, 12 bp, Artificial Sequence
  Description of Artificial Sequence Synthetic oligonucleotide
agaatcagtaga

SEQ ID NO: 5 - DNA, 12 bp, Artificial Sequence
  Description of Artificial Sequence Synthetic oligonucleotide
agactcagtaga

SEQ ID NO: 6 - DNA, 20 bp, Artificial Sequence
  Description of Artificial Sequence Synthetic oligonucleotide
cggcattccgtcagtcaaat

SEQ ID NO: 7 - DNA, 20 bp, Artificial Sequence
  Description of Artificial Sequence Synthetic oligonucleotide
agaatcagtagaggaaatta

SEQ ID NO: 8 - DNA, 19 bp, Artificial Sequence
  Description of Artificial Sequence Synthetic oligonucleotide
agactcagtagaggaaagt

SEQ ID NO: 9 - DNA, 82 bp, Artificial Sequence
  Description of Artificial Sequence Synthetic oligonucleotide
acggcatacgagattgacatgtgactggagttcagacgtgtgctcttccgatctatacctgtgacaaccagtgttgttccac

SEQ ID NO: 10 - DNA, 142 bp, Artificial Sequence
  Description of Artificial Sequence Synthetic polynucleotide
gccaaagagtttccctagcgcgggtgctcaatcccaggtggcacagtaatagaccccagagtcacgttcaattagattttccagtataaatttaaggctcttccctgtgcttgcataagtatgatacttttctcgactgatt

SEQ ID NO: 11 - DNA, 78 bp, Artificial Sequence
  Description of Artificial Sequence Synthetic oligonucleotide
ctgattccaacacatcccttgcggagatcggaagagcgtcgtgtagggaaagagtgtagatctcggtggtcgccgtat

SEQ ID NO: 12 - DNA, 142 bp, Artificial Sequence
  Description of Artificial Sequence Synthetic polynucleotide
  modified_base (74)..(74): a, c, t, g, unknown or other
gccaaagagtttccctagcgcgggtgctcaatcccaggtggcacagtaatagaccccagagtcacgttcaattngattttccagtataaatttaaggctcttccctgtgcttgcataagtatgatacttttctcgactgatt

SEQ ID NO: 13 - DNA, 263 bp, Artificial Sequence
  Description of Artificial Sequence Synthetic polynucleotide
gcctctggattcaccttcagtagctactggatgcactgggtccgccaagctccagggaaggggctggtgtgggtctcacgtattaatagtgatgggagtagcacaagctacgcggactccgtgaagggccgattcaccatctccagagacaacgccaagaacacgctgtatctgcaaatgaacagtctgagagccgaggacacggctgtgtattactgtgcaagagatagtaacggtatggacgtctggggccaagggaccac

SEQ ID NO: 14 - DNA, 259 bp, Artificial Sequence
  Description of Artificial Sequence Synthetic polynucleotide
cttctggttacacctttaccagctacggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagatcacccatttgactactggggccagggaaccct

SEQ ID NO: 15 - DNA, 269 bp, Artificial Sequence
  Description of Artificial Sequence Synthetic polynucleotide
gcctctggattcaccttcagtagctatgctatgcactgggtccgccaggctccaggcaaggggctggagtgggtggcagttatatcatatgatggaagcaataaatactacgcagactccgtgaagggccgattcaccatctccagagacaattccaagaacacgctgtatctgcaaatgaacagcctgagagctgaggacacggctgtgtattactgtgcgagagatggggaggaactgggatttgactactggggccagggaaccct

SEQ ID NO: 16 - DNA, 15 bp, Artificial Sequence
  Description of Artificial Sequence Synthetic oligonucleotide
cactgtctctggtgg

SEQ ID NO: 17 - DNA, 15 bp, Artificial Sequence
  Description of Artificial Sequence Synthetic oligonucleotide
gcctctggattcacc

SEQ ID NO: 18 - DNA, 15 bp, Artificial Sequence
  Description of Artificial Sequence Synthetic oligonucleotide
gcctccgaactcacc

SEQ ID NO: 19 - DNA, 15 bp, Artificial Sequence
  Description of Artificial Sequence Synthetic oligonucleotide
tgccatctccgggga

SEQ ID NO: 20 - DNA, 16 bp, Artificial Sequence
  Description of Artificial Sequence Synthetic oligonucleotide
gcctctggattcatct

SEQ ID NO: 21 - DNA, 15 bp, Artificial Sequence
  Description of Artificial Sequence Synthetic oligonucleotide
gcctctggtctcagc

SEQ ID NO: 22 - DNA, 15 bp, Artificial Sequence
  Description of Artificial Sequence Synthetic oligonucleotide
gcctctgggttcacc

SEQ ID NO: 23 - DNA, 15 bp, Artificial Sequence
  Description of Artificial Sequence Synthetic oligonucleotide
gcgtctggattcacc

SEQ ID NO: 24 - DNA, 289 bp, Artificial Sequence
  Description of Artificial Sequence Synthetic polynucleotide
cttccggttacacttttaggaattacggtattacctgggtgcgacaggcccctggacaggggcttgagtggatggcgttcatcagcgctttcaatgttaacacaaagtatgcacagaatttccaggacagagtcaccgttactacagacactgccacgagaacagttcacctggaggtgaggagcctcattcctgacgacacggccgtgtattattgtgcgaggtccagaatccccttcggtggtttatggagtggctatgctcttgatgtctggggccaagggacagt

SEQ ID NO: 25 - DNA, 16 bp, Artificial Sequence
  Description of Artificial Sequence Synthetic oligonucleotide
cttccggttacacttt

SEQ ID NO: 26 - DNA, 15 bp, Artificial Sequence
  Description of Artificial Sequence Synthetic oligonucleotide
gcctcaggattcacc

SEQ ID NO: 27 - DNA, 15 bp, Artificial Sequence
  Description of Artificial Sequence Synthetic oligonucleotide
gcctctggattcagc

SEQ ID NO: 28 - DNA, 15 bp, Artificial Sequence
  Description of Artificial Sequence Synthetic oligonucleotide
gcctctggattcaca

SEQ ID NO: 29 - DNA, 15 bp, Artificial Sequence
  Description of Artificial Sequence Synthetic oligonucleotide
gggttctccttgttg

SEQ ID NO: 30 - DNA, 15 bp, Artificial Sequence
  Description of Artificial Sequence Synthetic oligonucleotide
ctgggttctccttgt

(19) United States
(12) Patent Application Publication
(10) Pub. No.: US 2020/0385806 A1
(43) Pub. Date: Dec. 10, 2020

(54) SYSTEM AND METHODS FOR PRIMER EXTRACTION AND CLONALITY DETECTION

(71) Applicant: MEMORIAL SLOAN KETTERING CANCER CENTER, New York, NY (US)

(72) Inventors: Ahmet ZEHIR, New York, NY (US); Mustafa SYED, New York, NY (US); Maria ARCILA, New York, NY (US)

(21) Appl. No.: 16/755,102

(22) PCT Filed: Oct. 9, 2018
(86) PCT No.: PCT/US2018/055083

§ 371 Date: Apr. 9, 2020

Related U.S. Application Data
(60) Provisional application No. 62/570,549, filed on Oct. 10, 2017.
(60) Provisional application No. 62/700,794, filed on Jul. 19, 2018.

(51) Int. Cl. C12Q 1/6881; G16B 25/20; C12Q 1/6869; C12Q 1/6809

(52) U.S. Cl. CPC C12Q 1/6881; G16B 25/20; C12Q 2535/122; C12Q 1/6809; C12Q 1/6869

(57) ABSTRACT

A genomic data processing system can be configured to process next-generation sequencing information. In one embodiment, the genomic data processing system can determine forward and reverse primers from sequence reads provided by a next-generation sequencer. By determining forward and reverse primers, accuracy of the detection of clonality can be improved. In another embodiment, a genomic data processing system can be configured to detect clonalities in genetic data.

### CROSS-REFERENCE TO RELATED APPLICATIONS

This application is a National Stage Application of PCT/US2018/055083, filed Oct. 9, 2018, which claims the benefit of and priority to U.S. Provisional Patent Application No. 62/570,549, filed Oct. 10, 2017, and also to U.S. Provisional Patent Application No. 62/700,794, filed Jul. 19, 2018, the entire contents of each of which are incorporated herein by reference.

### SEQUENCE LISTING

The instant application contains a Sequence Listing which has been submitted electronically in ASCII format and is hereby incorporated by reference in its entirety. Said ASCII copy, created on Nov. 20, 2018, is named 115872-1930_SL.txt and is 9,172 bytes in size.

### FIELD OF THE DISCLOSURE

The present disclosure is generally directed to processing data to determine primers and detect clonality in genomic data.

### BACKGROUND OF THE DISCLOSURE

Genomic data processing can include detecting clonality using sequence reads received from a next-generation sequencer. Primers used to generate the sequence reads may not be readily available, making it difficult to determine the accuracy and of the sequence reads. In some instances, an accuracy of the next-generation sequencer for detecting clones may be affected by the primers used.

### BRIEF SUMMARY OF THE DISCLOSURE

In one aspect, the disclosure includes a computer-implemented method to identify at least one primer of assays utilized in next-generation sequencing of a sample. The method includes, generating, by a computer server including one or more processors, from genomic data received from the next generation sequencing device, a plurality of sequence reads derived from biological samples that have been processed with forward primers and reverse primers of a next generation sequencing assay. The method also includes generating, by the computer server, a plurality of V-J gene segments by performing a lookup of each sequence read in the plurality of sequence reads in a genome database. The method further includes comparing by the computer server, each V-J gene segment of the plurality of V-J gene segments with the genomic data received from the next generation sequencing device to identify for the corresponding V-J gene segment a first number of nucleotides located upstream of the corresponding V-J gene segment and a second number of nucleotides located downstream of the corresponding V-J gene segment. The method also includes grouping, by the computer server, the plurality of V-J gene segments into a plurality of groups, each group including V-J gene segments having a same V-J identity. The method further includes, for each group of the plurality of groups, aligning by the computer server, for the V-J gene segments within the group, respective second number of nucleotides located downstream of the V-J gene segment. The method further includes, for each group of the plurality of groups, aligning by the computer server, for the V-J gene segments within the group, respective first number of nucleotides located upstream of the V-J gene segment. The method further includes, for each group of the plurality of groups, determining by the computer server, for the aligned respective first number of nucleotides located upstream of the V-J gene segment, at each nucleotide position, a nucleotide identity corresponding to a consensus policy to generate a forward primer consensus sequence, and determining, by the computer server, for the aligned respective second number of nucleotides located downstream of the V-J gene segment, at each nucleotide position, a nucleotide identity corresponding to the consensus policy to generate a reverse primer consensus sequence. The method also includes identifying by the computer server, a plurality of forward primer consensus sequences as the forward primers of the next generation sequencing assay and identifying a plurality of reverse primer consensus sequences as the reverse primers of the next generation sequencing assay.

In some embodiments, at least one or more of the plurality of V-J gene segments further comprise a Diversity (D) region. In some embodiments, the biological sample comprises nucleic acids selected from the group consisting of DNA and RNA. In some embodiments, the nucleic acids are derived from one or more T lymphocytes selected from the group consisting of CD4+ helper T cells, CD8+ cytotoxic T cells, memory T cells, gamma-delta T cells, and regulatory T cells. In some embodiments, the nucleic acids are derived from one or more B lymphocytes selected from the group consisting of plasma cells, memory B cells, follicular B cells, marginal zone B cells, and regulatory B cells. In some embodiments, the biological sample is obtained from a patient that is diagnosed with, is suspected of having, or is at risk for a lymphoproliferative disorder. In some embodiments, the lymphoproferative disorder is leukemia, follicular lymphoma, chronic lymphocytic leukemia, acute lymphoblastic leukemia, hairy cell leukemia, B-cell lymphoma, T-cell lymphomas, multiple myeloma, Waldenstrom's macroglobulinemia, Wiskott-Aldrich syndrome, Lymphocyte-variant hypereosinophilia, post-transplant lymphoproliferative disorder, autoimmune lymphoproliferative syndrome (ALPS) or Lymphoid interstitial pneumonia.

In some embodiments, the assays utilized in next-generation sequencing of the sample are selected from the group consisting of IGH FR1 assay, IGH FR2 assay, IGH FR3 assay, IGHV leader somatic hypermutation assay, TRG assay, and IGK assay. In some embodiments, the reverse primers are between 20-30 base pairs in length. In some embodiments, the forward primers are between 20-30 base pairs in length. In some embodiments, the reverse primers and the forward primers further comprise a NGS-compatible adapter sequence. In some embodiments, the NGS-compatible adapter sequence is a P5 adapter, P7 adapter, P1 adapter, A adapter, or Ion Xpress™ barcode adapter. In some embodiments, the reverse primers comprise an adapter sequence that is distinct from the forward primers. In some embodiments, comparing each V-J gene segment of the plurality of V-J gene segments with the genomic data received from the next generation sequencing device includes comparing by the computer server, each V-J gene segment of the plurality of V-J gene segments to the plurality of sequence reads derived from biological samples.

In some embodiments, the method further includes accessing, by the computer server over a communication channel, the genome database to perform the lookup of each sequence read in the plurality of sequence reads in the genome database. In some embodiments, the method further includes storing, by the computer server in a first array data structure in memory, the first number of nucleotides located upstream of the V-J gene segment, one dimension of the first array data structure being indexed to a position of a nucleotide, determining, by the computer server at each position along the one dimension of the first array data structure, the nucleotide identity corresponding to the consensus policy, and generating, by the computer server, the forward primer consensus sequence based on the nucleotide identities determined for at least two positions along the one dimension of the first array data structure.

In some embodiments, the method further includes storing, by the computer server in a second array data structure in memory, the second number of nucleotides located downstream of the V-J gene segment, one dimension of the second array data structure being indexed to a position of a nucleotide, determining, by the computer server at each position along the one dimension of the second array data structure, the nucleotide identity corresponding to the consensus policy, and generating, by the computer server, the reverse primer consensus sequence based on the nucleotide identities determined for at least two positions along the one dimension of the second array data structure.

In one aspect, the disclosure includes a system including one or more processors, and a memory coupled to the one or more processors, the memory storing computer-executable instructions, which when executed by the one or more processors, causes the one or more processors to generate, from genomic data received from the next generation sequencing device, a plurality of sequence reads derived from biological samples that have been processed with forward primers and reverse primers of a next generation sequencing assay. The instructions causes the one or more processor to further generate a plurality of V-J gene segments by performing a lookup of each sequence read in the plurality of sequence reads in a genome database, and compare each V-J gene segment of the plurality of V-J gene segments with the genomic data received from the next generation sequencing device to identify for the corresponding V-J gene segment a first number of nucleotides located upstream of the corresponding V-J gene segment and a second number of nucleotides located downstream of the corresponding V-J gene segment. The instructions causes the one or more processor to further group the plurality of V-J gene segments into a plurality of groups, each group including V-J gene segments having a same V-J identity, and for each group of the plurality of groups: align, for the V-J gene segments within the group, respective second number of nucleotides located downstream of the V-J gene segment, align, for the V-J gene segments within the group, respective first number of nucleotides located upstream of the V-J gene segment, determine, for the aligned respective first number of nucleotides located upstream of the V-J gene segment, at each nucleotide position, a nucleotide identity corresponding to a consensus policy to generate a forward primer consensus sequence, determine, for the aligned respective second number of nucleotides located downstream of the V-J gene segment, at each nucleotide position, a nucleotide identity corresponding to the consensus policy to generate a reverse primer consensus sequence, and identify a plurality of forward primer consensus sequences as the forward primers of the next generation sequencing assay and identifying a plurality of reverse primer consensus sequences as the reverse primers of the next generation sequencing assay.

In some embodiments, at least one or more of the plurality of V-J gene segments further comprise a Diversity (D) region. In some embodiments, the biological sample comprises nucleic acids selected from the group consisting of DNA and RNA. In some embodiments, the nucleic acids are derived from one or more T lymphocytes selected from the group consisting of CD4+ helper T cells, CD8+ cytotoxic T cells, memory T cells, gamma-delta T cells, and regulatory T cells. In some embodiments the nucleic acids are derived from one or more B lymphocytes selected from the group consisting of plasma cells, memory B cells, follicular B cells, marginal zone B cells, and regulatory B cells. In some embodiments, the biological sample is obtained from a patient that is diagnosed with, is suspected of having, or is at risk for a lymphoproliferative disorder. In some embodiments, the lymphoproferative disorder is leukemia, follicular lymphoma, chronic lymphocytic leukemia, acute lymphoblastic leukemia, hairy cell leukemia, B-cell lymphoma, T-cell lymphomas, multiple myeloma, Waldenstrom's macroglobulinemia, Wiskott-Aldrich syndrome, Lymphocyte-variant hypereosinophilia, post-transplant lymphoproliferative disorder, autoimmune lymphoproliferative syndrome (ALPS) or Lymphoid interstitial pneumonia. In some embodiments, the assays utilized in next-generation sequencing of the sample are selected from the group consisting of IGH FR1 assay, IGH FR2 assay, IGH FR3 assay, IGHV leader somatic hypermutation assay, TRG assay, and IGK assay.

In some embodiments, the reverse primers are between 20-30 base pairs in length. In some embodiments, the forward primers are between 20-30 base pairs in length. In some embodiments, the reverse primers and the forward primers further comprise a NGS-compatible adapter sequence. In some embodiments, the NGS-compatible adapter sequence is a P5 adapter, P7 adapter, P1 adapter, A adapter, or Ion Xpress™ barcode adapter. In some embodiments, the reverse primers comprise an adapter sequence that is distinct from the forward primers. In some embodiments, comparing each V-J gene segment of the plurality of V-J gene segments with the genomic data received from the next generation sequencing device includes comparing by the computer server, each V-J gene segment of the plurality of V-J gene segments to the plurality of sequence reads derived from biological samples.

In some embodiments, the memory storing computer-executable instructions, which when executed by the one or more processors, causes the one or more processors to: access, by the computer server over a communication channel, the genome database to perform the lookup of each sequence read in the plurality of sequence reads in the genome database. In some embodiments, the memory storing computer-executable instructions, which when executed by the one or more processors, causes the one or more processors to: store, by the computer server in a first array data structure in memory, the first number of nucleotides located upstream of the V-J gene segment, one dimension of the first array data structure being indexed to a position of a nucleotide, determine, by the computer server at each position along the one dimension of the first array data structure, the nucleotide identity corresponding to the consensus policy, and generate, by the computer server, the forward primer consensus sequence based on the nucleotide identities determined for at least two positions along the one dimension of the first array data structure.

In some embodiments, the memory storing computer-executable instructions, which when executed by the one or more processors, causes the one or more processors to: store, by the computer server in a second array data structure in memory, the second number of nucleotides located downstream of the V-J gene segment, one dimension of the second array data structure being indexed to a position of a nucleotide, determine, by the computer server at each position along the one dimension of the second array data structure, the nucleotide identity corresponding to the consensus policy, and generate, by the computer server, the reverse primer consensus sequence based on the nucleotide identities determined for at least two positions along the one dimension of the second array data structure.

In one aspect, the disclosure includes a computer readable storage medium storing processor-executable instructions which, when executed by the at least one processor, causes the at least one processor to generate, from genomic data received from the next generation sequencing device, a plurality of sequence reads derived from biological samples that have been processed with forward primers and reverse primers of a next generation sequencing assay. The instructions cause the one or more processors to generate a plurality of V-J gene segments by performing a lookup of each sequence read in the plurality of sequence reads in a genome database, and compare each V-J gene segment of the plurality of V-J gene segments with the genomic data received from the next generation sequencing device to identify for the corresponding V-J gene segment a first number of nucleotides located upstream of the corresponding V-J gene segment and a second number of nucleotides located downstream of the corresponding V-J gene segment. The instructions cause the one or more processors to group the plurality of V-J gene segments into a plurality of groups, each group including V-J gene segments having a same V-J identity, for each group of the plurality of groups: align, for the V-J gene segments within the group, respective second number of nucleotides located downstream of the V-J gene segment, align, for the V-J gene segments within the group, respective first number of nucleotides located upstream of the V-J gene segment, determine, for the aligned respective first number of nucleotides located upstream of the V-J gene segment, at each nucleotide position, a nucleotide identity corresponding to a consensus policy to generate a forward primer consensus sequence, determine, for the aligned respective second number of nucleotides located downstream of the V-J gene segment, at each nucleotide position, a nucleotide identity corresponding to the consensus policy to generate a reverse primer consensus sequence, and identify a plurality of forward primer consensus sequences as the forward primers of the next generation sequencing assay and identifying a plurality of reverse primer consensus sequences as the reverse primers of the next generation sequencing assay.

In some embodiments, at least one or more of the plurality of V-J gene segments further comprise a Diversity (D) region. In some embodiments, the biological sample comprises nucleic acids selected from the group consisting of DNA and RNA. In some embodiments, the nucleic acids are derived from one or more T lymphocytes selected from the group consisting of CD4+ helper T cells, CD8+ cytotoxic T cells, memory T cells, gamma-delta T cells, and regulatory T cells. In some embodiments, the nucleic acids are derived from one or more B lymphocytes selected from the group consisting of plasma cells, memory B cells, follicular B cells, marginal zone B cells, and regulatory B cells. In some embodiments, the biological sample is obtained from a patient that is diagnosed with, is suspected of having, or is at risk for a lymphoproliferative disorder. In some embodiments, the lymphoproferative disorder is leukemia, follicular lymphoma, chronic lymphocytic leukemia, acute lymphoblastic leukemia, hairy cell leukemia, B-cell lymphoma, T-cell lymphomas, multiple myeloma, Waldenstrom's macroglobulinemia, Wiskott-Aldrich syndrome, Lymphocyte-variant hypereosinophilia, post-transplant lymphoproliferative disorder, autoimmune lymphoproliferative syndrome (ALPS) or Lymphoid interstitial pneumonia. In some embodiments, the assays utilized in next-generation sequencing of the sample are selected from the group consisting of IGH FR1 assay, IGH FR2 assay, IGH FR3 assay, IGHV leader somatic hypermutation assay, TRG assay, and IGK assay.

In some embodiments, the reverse primers are between 20-30 base pairs in length. In some embodiments, the forward primers are between 20-30 base pairs in length. In some embodiments, the reverse primers and the forward primers further comprise a NGS-compatible adapter sequence. In some embodiments, the NGS-compatible adapter sequence is a P5 adapter, P7 adapter, P1 adapter, A adapter, or Ion Xpress™ barcode adapter. In some embodiments, the reverse primers comprise an adapter sequence that is distinct from the forward primers. In some embodiments, comparing each V-J gene segment of the plurality of V-J gene segments with the genomic data received from the next generation sequencing device includes comparing by the computer server, each V-J gene segment of the plurality of V-J gene segments to the plurality of sequence reads derived from biological samples. In some embodiments, the instructions causing the one or more processors to: access, by the computer server over a communication channel, the genome database to perform the lookup of each sequence read in the plurality of sequence reads in the genome database.

In some embodiments, the instructions causing the one or more processors to store, by the computer server in a first array data structure in memory, the first number of nucleotides located upstream of the V-J gene segment, one dimension of the first array data structure being indexed to a position of a nucleotide, determine, by the computer server at each position along the one dimension of the first array data structure, the nucleotide identity corresponding to the consensus policy, and generate, by the computer server, the forward primer consensus sequence based on the nucleotide identities determined for at least two positions along the one dimension of the first array data structure.

In some embodiments, the instructions causing the one or more processors to: store, by the computer server in a second array data structure in memory, the second number of nucleotides located downstream of the V-J gene segment, one dimension of the second array data structure being indexed to a position of a nucleotide, determine, by the computer server at each position along the one dimension of the second array data structure, the nucleotide identity corresponding to the consensus policy, and generate, by the computer server, the reverse primer consensus sequence based on the nucleotide identities determined for at least two positions along the one dimension of the second array data structure.

In one aspect, the disclosure includes a computer-implemented method for detecting at least one clonal V-J gene segment in samples obtained from subjects. The method includes receiving, by a computer server including one or more processors, from a next generation sequencing device, a plurality of sequence reads associated with a sample obtained from a subject, each sequence read representing at least one of coding gene segments or non-coding gene segments. The method also includes removing, by the computer server, for each sequence read of the plurality of sequence reads, a respective forward primer sequence and a respective reverse primer sequence to generate a corresponding trimmed sequence read. The method further includes identifying, by the computer server, from trimmed sequence reads generated from the plurality of sequence reads, a plurality of groups of trimmed sequence reads, each group including trimmed sequence reads having a same sequence identity. The method also includes select, by the computer server, one trimmed sequence read from each of the plurality of groups to form a selected set of trimmed sequence reads. The method further includes determining, by the computer server, for each trimmed sequence read in the selected set of trimmed sequence reads, a V-J identity by comparing the trimmed sequence read to a human genome database that includes associations between nucleotide sequences and V-J identities. The method additionally includes determining, by the computer server, for each V-J identity corresponding to a group of the plurality of groups of trimmed sequence reads, a respective frequency of the V-J identity based on a number of trimmed sequence reads included in the group. The method also includes identifying, by the computer server, based on the respective frequency of the V-J identity corresponding to a first group of the plurality of groups of trimmed sequence reads, at least one clone of the V-J identity based on a clonal detection policy.

In some embodiments, the at least one clonal V-J gene segment further comprise a Diversity (D) region. In some embodiments, the biological samples comprise nucleic acids selected from the group consisting of DNA and RNA. In some embodiments, the nucleic acids are derived from one or more T lymphocytes selected from the group consisting of CD4+ helper T cells, CD8+ cytotoxic T cells, memory T cells, gamma-delta T cells, and regulatory T cells. In some embodiments, the nucleic acids are derived from one or more B lymphocytes selected from the group consisting of plasma cells, memory B cells, follicular B cells, marginal zone B cells, and regulatory B cells. In some embodiments, the subjects are diagnosed with, are suspected of having, or are at risk for a lymphoproliferative disorder.

In some embodiments, the lymphoproferative disorder is leukemia, follicular lymphoma, chronic lymphocytic leukemia, acute lymphoblastic leukemia, hairy cell leukemia, B-cell lymphoma, T-cell lymphomas, multiple myeloma, Waldenstrom's macroglobulinemia, Wiskott-Aldrich syndrome, Lymphocyte-variant hypereosinophilia, post-transplant lymphoproliferative disorder, autoimmune lymphoproliferative syndrome (ALPS) or Lymphoid interstitial pneumonia. In some embodiments, the respective reverse primer sequence of each sequence read is between 20-30 base pairs in length. In some embodiments, the respective forward primer sequence of each sequence read is between 20-30 base pairs in length. In some embodiments, the respective forward primer sequence and the respective reverse primer sequence of each sequence read further comprise a NGS-compatible adapter sequence. In some embodiments, the NGS-compatible adapter sequence is a P5 adapter, P7 adapter, P1 adapter, A adapter, or Ion Xpress™ barcode adapter. In some embodiments, the respective forward primer sequence and the respective reverse primer sequence of each sequence read comprise distinct NGS-compatible adapter sequences.

In one aspect, the disclosure includes a system having one or more processors. The system further includes a memory coupled to the one or more processors, the memory storing computer-executable instructions, which when executed by the one or more processors, causes the one or more processors to receive, by a computer server including one or more processors, from a next generation sequencing device, a plurality of sequence reads associated with a sample obtained from a subject, each sequence read representing at least one of coding gene segments or non-coding gene segments. The instructions causes the one or more processor to remove, by the computer server, for each sequence read of the plurality of sequence reads, a respective forward primer sequence and a respective reverse primer sequence to generate a corresponding trimmed sequence read, and identify, by the computer server, from trimmed sequence reads generated from the plurality of sequence reads, a plurality of groups of trimmed sequence reads, each group including trimmed sequence reads having a same sequence identity. The instructions causes the one or more processor to select, by the computer server, one trimmed sequence read from each of the plurality of groups to form a selected set of trimmed sequence reads, determine, by the computer server, for each trimmed sequence read in the selected set of trimmed sequence reads, a V-J identity by comparing the trimmed sequence read to a human genome database that includes associations between nucleotide sequences and V-J identities. The instructions causes the one or more processor to determine, by the computer server, for each V-J identity corresponding to a group of the plurality of groups of trimmed sequence reads, a respective frequency of the V-J identity based on a number of trimmed sequence reads included in the group, and identify, by the computer server, based on the respective frequency of the V-J identity corresponding to a first group of the plurality of groups of trimmed sequence reads, at least one clone of the V-J identity based on a clonal detection policy.

In some embodiments, the at least one clonal V-J gene segment further comprise a Diversity (D) region. In some embodiments, the biological samples comprise nucleic acids selected from the group consisting of DNA and RNA. In some embodiments, the nucleic acids are derived from one or more T lymphocytes selected from the group consisting of CD4+ helper T cells, CD8+ cytotoxic T cells, memory T cells, gamma-delta T cells, and regulatory T cells. In some embodiments, the nucleic acids are derived from one or more B lymphocytes selected from the group consisting of plasma cells, memory B cells, follicular B cells, marginal zone B cells, and regulatory B cells. In some embodiments, the subjects are diagnosed with, are suspected of having, or are at risk for a lymphoproliferative disorder.

In some embodiments, the lymphoproferative disorder is leukemia, follicular lymphoma, chronic lymphocytic leukemia, acute lymphoblastic leukemia, hairy cell leukemia, B-cell lymphoma, T-cell lymphomas, multiple myeloma, Waldenstrom's macroglobulinemia, Wiskott-Aldrich syndrome, Lymphocyte-variant hypereosinophilia, post-transplant lymphoproliferative disorder, autoimmune lymphoproliferative syndrome (ALPS) or Lymphoid interstitial pneumonia. In some embodiments, the respective reverse primer sequence of each sequence read is between 20-30 base pairs in length. In some embodiments, the respective forward primer sequence of each sequence read is between 20-30 base pairs in length. In some embodiments, the respective forward primer sequence and the respective reverse primer sequence of each sequence read further comprise a NGS-compatible adapter sequence. In some embodiments, the NGS-compatible adapter sequence is a P5 adapter, P7 adapter, P1 adapter, A adapter, or Ion Xpress barcode adapter. In some embodiments, the respective forward primer sequence and the respective reverse primer sequence of each sequence read comprise distinct NGS-compatible adapter sequences.

In one aspect the disclosure includes a computer readable storage medium storing processor-executable instructions which, when executed by the at least one processor, causes the at least one processor to receive, by a computer server including one or more processors, from a next generation sequencing device, a plurality of sequence reads associated with a sample obtained from a subject, each sequence read representing at least one of coding gene segments or non-coding gene segments. The instructions causes the at least one processor to remove, by the computer server, for each sequence read of the plurality of sequence reads, a respective forward primer sequence and a respective reverse primer sequence to generate a corresponding trimmed sequence read, and identify, by the computer server, from trimmed sequence reads generated from the plurality of sequence reads, a plurality of groups of trimmed sequence reads, each group including trimmed sequence reads having a same sequence identity. The instructions causes the at least one processor to select, by the computer server, one trimmed sequence read from each of the plurality of groups to form a selected set of trimmed sequence reads, and determine, by the computer server, for each trimmed sequence read in the selected set of trimmed sequence reads, a V-J identity by comparing the trimmed sequence read to a human genome database that includes associations between nucleotide sequences and V-J identities. The instructions causes the at least one processor to determine, by the computer server, for each V-J identity corresponding to a group of the plurality of groups of trimmed sequence reads, a respective frequency of the V-J identity based on a number of trimmed sequence reads included in the group, and identify, by the computer server, based on the respective frequency of the V-J identity corresponding to a first group of the plurality of groups of trimmed sequence reads, at least one clone of the V-J identity based on a clonal detection policy.

In some embodiments, the at least one clonal V-J gene segment further comprise a Diversity (D) region. In some embodiments, the biological samples comprise nucleic acids selected from the group consisting of DNA and RNA. In some embodiments, the nucleic acids are derived from one or more T lymphocytes selected from the group consisting of CD4+ helper T cells, CD8+ cytotoxic T cells, memory T cells, gamma-delta T cells, and regulatory T cells. In some embodiments, the nucleic acids are derived from one or more B lymphocytes selected from the group consisting of plasma cells, memory B cells, follicular B cells, marginal zone B cells, and regulatory B cells. In some embodiments, the subjects are diagnosed with, are suspected of having, or are at risk for a lymphoproliferative disorder.

In some embodiments, the lymphoproferative disorder is leukemia, follicular lymphoma, chronic lymphocytic leukemia, acute lymphoblastic leukemia, hairy cell leukemia, B-cell lymphoma, T-cell lymphomas, multiple myeloma, Waldenstrom's macroglobulinemia, Wiskott-Aldrich syndrome, Lymphocyte-variant hypereosinophilia, post-transplant lymphoproliferative disorder, autoimmune lymphoproliferative syndrome (ALPS) or Lymphoid interstitial pneumonia. In some embodiments, the respective reverse primer sequence of each sequence read is between 20-30 base pairs in length. In some embodiments, the respective forward primer sequence of each sequence read is between 20-30 base pairs in length. In some embodiments, the respective forward primer sequence and the respective reverse primer sequence of each sequence read further comprise a NGS-compatible adapter sequence. In some embodiments, the NGS-compatible adapter sequence is a P5 adapter, P7 adapter, P1 adapter, A adapter, or Ion Xpress™ barcode adapter. In some embodiments, the respective forward primer sequence and the respective reverse primer sequence of each sequence read comprise distinct NGS-compatible adapter sequences.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1A is a block diagram depicting an embodiment of a network environment comprising a client device in communication with server device;

FIG. 1B is a block diagram depicting a cloud computing environment comprising client device in communication with cloud service providers;

FIGS. 1C and 1D are block diagrams depicting embodiments of computing devices useful in connection with the methods and systems described herein.

FIG. 2 illustrates a genomic data processing system.

FIG. 3 illustrates a flow diagram of a primer extraction process.

FIG. 4 illustrates screenshots of generating example sequence reads from genomic data provided by an example next generation sequencer. FIG. 4 discloses SEQ ID NOS 1-2 in the left column, SEQ ID NOS 1, 3, 3-4, 4, 4-5, 4, 4, 4-5, 4, 4-5, 4, 4-5, 5, 4, 4-5, 4, 4-5, 5, 4, 4, 3-4, 3, 3-4, 3-4, 4, 4 and 4 in the middle column and SEQ ID NOS 1-2, 6, 7, 1, 1, 8, 7, 1, 1, 8, 1, 7-8, 1, 7-8, 8, 1, 7-8, 1, 1, 8, 8, 1, 1-2, 1-2, 2, 1-2, 1, 1, 7 and 1 in the right column, all respectively, from top to bottom.

FIG. 5 shows one example of identifying a first number and a second number of nucleotides located upstream and downstream, respectively, of each V-J gene segment. FIG. 5 discloses the “Run4-TCR-349-25082” sequence as SEQ ID NOS 9-11, respectively, in order of appearance and the “lymphotrack” sequence as SEQ ID NO: 12.

FIG. 6 illustrates an alignment of the first number of nucleotides associated with V-J gene segments within a group.

FIG. 7 illustrates another genomic data processing system.

FIG. 8 illustrates a flow diagram of a clonal detection process.

FIG. 9 shows an example representation of forward and reverse primers for a plurality of sequence reads.

FIG. 10 shows an example representation of identifying a plurality of groups of trimmed sequence reads.

FIG. 11 shows an example output generated by a clonal detection engine.

FIG. 12 illustrates a set of clonal detection policies.

FIG. 13 illustrates follow-up data related to clone follow-up process. FIG. 13 discloses SEQ ID NOS 13-15, respectively, in order of appearance.

FIG. 14 illustrates a user interface for displaying the clones associated with a patient after a clone follow-up process.

FIGS. 15A-15E show a comparison between the clonal detection results achieved using the conventional Lymphotrack® Data Analysis Tool and the clonal detection process shown in FIG. 8. FIG. 15A discloses SEQ ID NOS 16-17, 17, 17-20, 17, 17, 17, 16, 21-22, 17, 17, 17 and 23, respectively, in order of appearance. FIG. 15B discloses SEQ ID NOS 24-25, 17, 17, 17, 17, 26, 17, 16-17, 17, 17, 17, 17, 22, 17, 27 and 17, respectively, in order of appearance.

FIG. 15D discloses SEQ ID NOS 23, 17, 28, 23, 23, 27, 16-17, 17, 17, 17, 17 and 17, respectively, in order of appearance. FIG. 15E discloses SEQ ID NOS 29-30, 30, 29, 29, 29, 29, 29, 29, 29, 29, 29-30, 29, 29-30 and 29-30, respectively, in order of appearance.

FIG. 16 shows the polyclonal distribution of various V-J gene rearrangements (e.g., >200 unique clones) observed in a sample derived from a normal control patient and a prominent peak representing a single population of a V-J gene rearrangement of particular length and sequence in a clonal sample. The different V-J gene rearrangements are represented by different colors.

### DETAILED DESCRIPTION

For purposes of reading the description of the various embodiments below, the following descriptions of the sections of the specification and their respective contents may be helpful:

Section A describes a network environment and computing environment which may be useful for practicing embodiments described herein.

Section B describes embodiments of systems and methods for identifying forward and reverse primers from genomic data.

Section C describes embodiments of systems and methods for detecting clonality in genomic data.

A. Computing and Network Environment

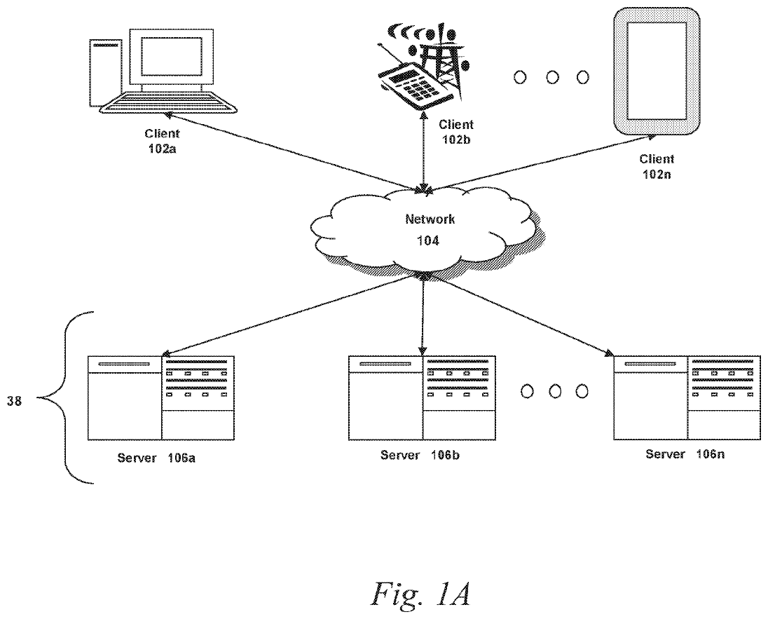

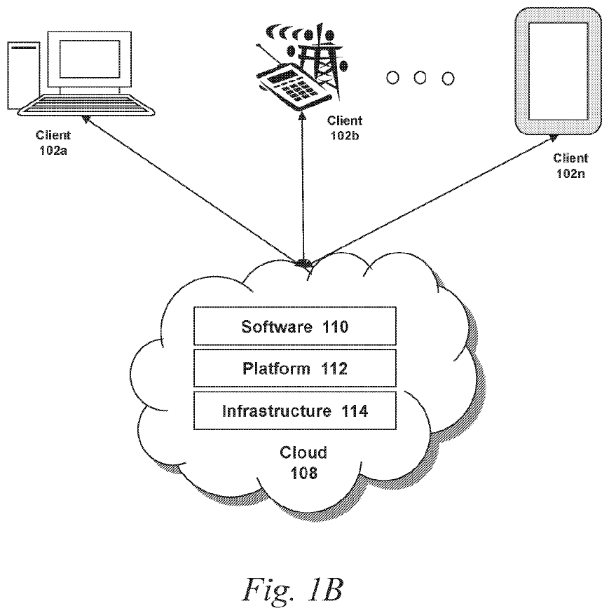

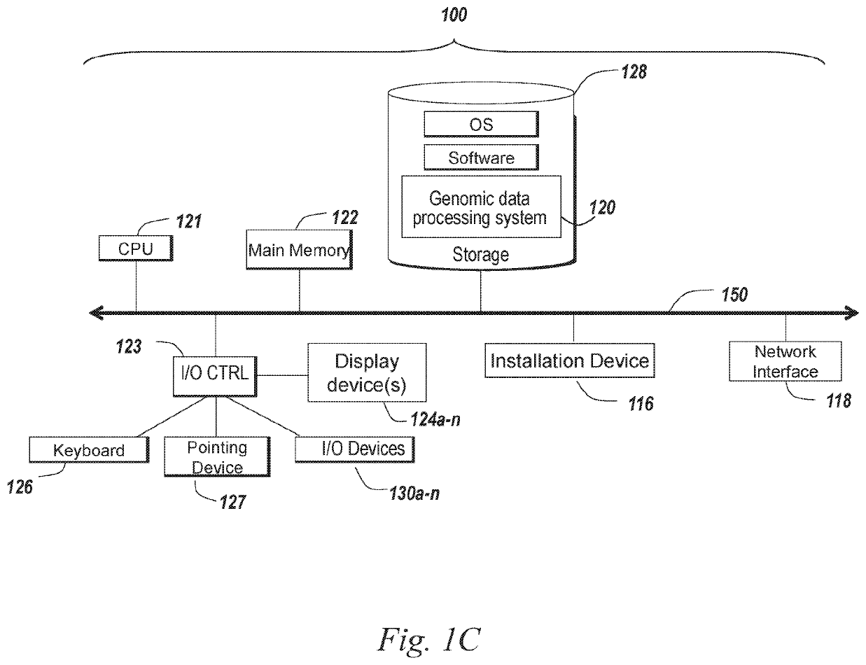

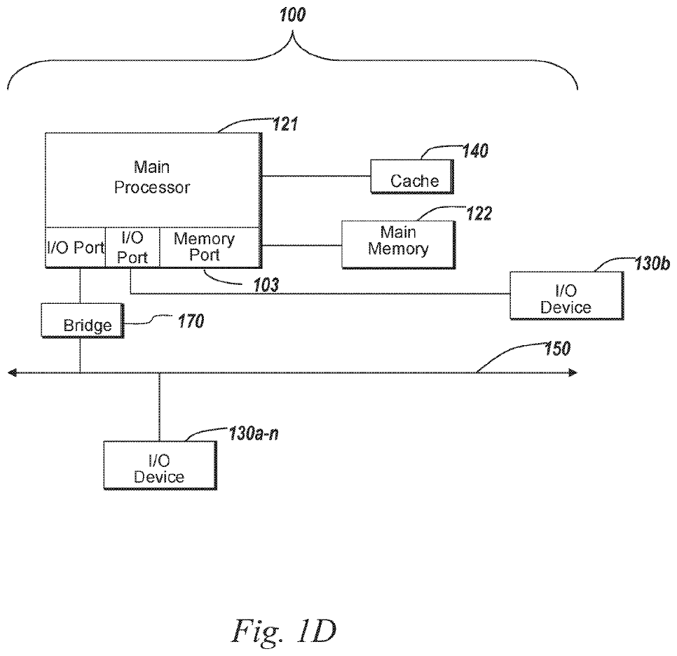

Prior to discussing specific embodiments of the present solution, it may be helpful to describe aspects of the operating environment as well as associated system components (e.g., hardware elements) in connection with the methods and systems described herein. Referring to FIG. 1A, an embodiment of a network environment is depicted. In brief overview, the network environment includes one or more clients 102a-102n (also generally referred to as local machine(s) 102, client(s) 102, client node(s) 102, client machine(s) 102, client computer(s) 102, client device(s) 102, endpoint(s) 102, or endpoint node(s) 102) in communication with one or more servers 106a-106n (also generally referred to as server(s) 106, node 106, or remote machine(s) 106) via one or more networks 104. In some embodiments, a client 102 has the capacity to function as both a client node seeking access to resources provided by a server and as a server providing access to hosted resources for other clients 102a-102n.

Although FIG. 1A shows a network 104 between the clients 102 and the servers 106, the clients 102 and the servers 106 may be on the same network 104. In some embodiments, there are multiple networks 104 between the clients 102 and the servers 106. In one of these embodiments, a network 104′ (not shown) may be a private network and a network 104 may be a public network. In another of these embodiments, a network 104 may be a private network and a network 104′ a public network. In still another of these embodiments, networks 104 and 104′ may both be private networks.

The network 104 may be connected via wired or wireless links. Wired links may include Digital Subscriber Line (DSL), coaxial cable lines, or optical fiber lines. The wireless links may include BLUETOOTH, Wi-Fi, Worldwide Interoperability for Microwave Access (WiMAX), an infrared channel or satellite band. The wireless links may also include any cellular network standards used to communicate among mobile devices, including standards that qualify as 1G, 2G, 3G, or 4G. The network standards may qualify as one or more generation of mobile telecommunication standards by fulfilling a specification or standards such as the specifications maintained by International Telecommunication Union. The 3G standards, for example, may correspond to the International Mobile Telecommunications-2000 (IMT-2000) specification, and the 4G standards may correspond to the International Mobile Telecommunications Advanced (IMT-Advanced) specification. Examples of cellular network standards include AMPS, GSM, GPRS, UMTS, LTE, LTE Advanced, Mobile WiMAX, and WiMAX-Advanced. Cellular network standards may use various channel access methods e.g. FDMA, TDMA, CDMA, or SDMA. In some embodiments, different types of data may be transmitted via different links and standards. In other embodiments, the same types of data may be transmitted via different links and standards.

The network 104 may be any type and/or form of network. The geographical scope of the network 104 may vary widely and the network 104 can be a body area network (BAN), a personal area network (PAN), a local-area network (LAN), e.g. Intranet, a metropolitan area network (MAN), a wide area network (WAN), or the Internet. The topology of the network 104 may be of any form and may include, e.g., any of the following: point-to-point, bus, star, ring, mesh, or tree. The network 104 may be an overlay network which is virtual and sits on top of one or more layers of other networks 104′. The network 104 may be of any such network topology as known to those ordinarily skilled in the art capable of supporting the operations described herein. The network 104 may utilize different techniques and layers or stacks of protocols, including, e.g., the Ethernet protocol, the internet protocol suite (TCP/IP), the ATM (Asynchronous Transfer Mode) technique, the SONET (Synchronous Optical Networking) protocol, or the SDH (Synchronous Digital Hierarchy) protocol. The TCP/IP internet protocol suite may include application layer, transport layer, internet layer (including, e.g., IPv6), or the link layer. The network 104 may be a type of a broadcast network, a telecommunications network, a data communication network, or a computer network.

In some embodiments, the system may include multiple, logically-grouped servers 106. In one of these embodiments, the logical group of servers may be referred to as a server farm 38 or a machine farm 38. In another of these embodiments, the servers 106 may be geographically dispersed. In other embodiments, a machine farm 38 may be administered as a single entity. In still other embodiments, the machine farm 38 includes a plurality of machine farms 38. The servers 106 within each machine farm 38 can be heterogeneous—one or more of the servers 106 or machines 106 can operate according to one type of operating system platform (e.g., WINDOWS NT, manufactured by Microsoft Corp. of Redmond, Wash.), while one or more of the other servers 106 can operate on according to another type of operating system platform (e.g., Unix, Linux, or Mac OS X).

In one embodiment, servers 106 in the machine farm 38 may be stored in high-density rack systems, along with associated storage systems, and located in an enterprise data center. In this embodiment, consolidating the servers 106 in this way may improve system manageability, data security, the physical security of the system, and system performance by locating servers 106 and high performance storage systems on localized high performance networks. Centralizing the servers 106 and storage systems and coupling them with advanced system management tools allows more efficient use of server resources.

The servers 106 of each machine farm 38 do not need to be physically proximate to another server 106 in the same machine farm 38. Thus, the group of servers 106 logically grouped as a machine farm 38 may be interconnected using a wide-area network (WAN) connection or a metropolitan-area network (MAN) connection. For example, a machine farm 38 may include servers 106 physically located in different continents or different regions of a continent, country, state, city, campus, or room. Data transmission speeds between servers 106 in the machine farm 38 can be increased if the servers 106 are connected using a local-area network (LAN) connection or some form of direct connection. Additionally, a heterogeneous machine farm 38 may include one or more servers 106 operating according to a type of operating system, while one or more other servers 106 execute one or more types of hypervisors rather than operating systems. In these embodiments, hypervisors may be used to emulate virtual hardware, partition physical hardware, virtualize physical hardware, and execute virtual machines that provide access to computing environments, allowing multiple operating systems to run concurrently on a host computer. Native hypervisors may run directly on the host computer. Hypervisors may include VMware ESX/ESXi, manufactured by VMWare, Inc., of Palo Alto, Calif.; the Xen hypervisor, an open source product whose development is overseen by Citrix Systems, Inc.; the HYPER-V hypervisors provided by Microsoft or others. Hosted hypervisors may run within an operating system on a second software level. Examples of hosted hypervisors may include VMware Workstation and VIRTUALBOX.

Management of the machine farm 38 may be de-centralized. For example, one or more servers 106 may comprise components, subsystems and modules to support one or more management services for the machine farm 38. In one of these embodiments, one or more servers 106 provide functionality for management of dynamic data, including techniques for handling failover, data replication, and increasing the robustness of the machine farm 38. Each server 106 may communicate with a persistent store and, in some embodiments, with a dynamic store.

Server 106 may be a file server, application server, web server, proxy server, appliance, network appliance, gateway, gateway server, virtualization server, deployment server, SSL VPN server, or firewall. In one embodiment, the server 106 may be referred to as a remote machine or a node. In another embodiment, a plurality of nodes 290 may be in the path between any two communicating servers.

Referring to FIG. 1B, a cloud computing environment is depicted. A cloud computing environment may provide client 102 with one or more resources provided by a network environment. The cloud computing environment may include one or more clients 102a-102n, in communication with the cloud 108 over one or more networks 104. Clients 102 may include, e.g., thick clients, thin clients, and zero clients. A thick client may provide at least some functionality even when disconnected from the cloud 108 or servers 106. A thin client or a zero client may depend on the connection to the cloud 108 or server 106 to provide functionality. A zero client may depend on the cloud 108 or other networks 104 or servers 106 to retrieve operating system data for the client device. The cloud 108 may include back end platforms, e.g., servers 106, storage, server farms or data centers.

The cloud 108 may be public, private, or hybrid. Public clouds may include public servers 106 that are maintained by third parties to the clients 102 or the owners of the clients. The servers 106 may be located off-site in remote geographical locations as disclosed above or otherwise. Public clouds may be connected to the servers 106 over a public network. Private clouds may include private servers 106 that are physically maintained by clients 102 or owners of clients. Private clouds may be connected to the servers 106 over a private network 104. Hybrid clouds 108 may include both the private and public networks 104 and servers 106.

The cloud 108 may also include a cloud based delivery, e.g. Software as a Service (SaaS) 110, Platform as a Service (PaaS) 112, and Infrastructure as a Service (IaaS) 114. IaaS may refer to a user renting the use of infrastructure resources that are needed during a specified time period. IaaS providers may offer storage, networking, servers or virtualization resources from large pools, allowing the users to quickly scale up by accessing more resources as needed. Examples of IaaS can include infrastructure and services (e.g., EG-32) provided by OVH HOSTING of Montreal, Quebec, Canada, AMAZON WEB SERVICES provided by Amazon.com, Inc., of Seattle, Wash., RACKSPACE CLOUD provided by Rackspace US, Inc., of San Antonio, Tex., Google Compute Engine provided by Google Inc. of Mountain View, Calif., or RIGHTSCALE provided by RightScale, Inc., of Santa Barbara, Calif. PaaS providers may offer functionality provided by IaaS, including, e.g., storage, networking, servers or virtualization, as well as additional resources such as, e.g., the operating system, middleware, or runtime resources. Examples of PaaS include WINDOWS AZURE provided by Microsoft Corporation of Redmond, Wash., Google App Engine provided by Google Inc., and HEROKU provided by Heroku, Inc. of San Francisco, Calif. SaaS providers may offer the resources that PaaS provides, including storage, networking, servers, virtualization, operating system, middleware, or runtime resources. In some embodiments, SaaS providers may offer additional resources including, e.g., data and application resources. Examples of SaaS include GOOGLE APPS provided by Google Inc., SALESFORCE provided by Salesforce.com Inc. of San Francisco, Calif., or OFFICE 365 provided by Microsoft Corporation. Examples of SaaS may also include data storage providers, e.g. DROPBOX provided by Dropbox, Inc. of San Francisco, Calif., Microsoft SKYDRIVE provided by Microsoft Corporation, Google Drive provided by Google Inc., or Apple ICLOUD provided by Apple Inc. of Cupertino, Calif.

Clients 102 may access IaaS resources with one or more IaaS standards, including, e.g., Amazon Elastic Compute Cloud (EC2), Open Cloud Computing Interface (OCCI), Cloud Infrastructure Management Interface (CIMI), or OpenStack standards. Some IaaS standards may allow clients access to resources over HTTP, and may use Representational State Transfer (REST) protocol or Simple Object Access Protocol (SOAP). Clients 102 may access PaaS resources with different PaaS interfaces. Some PaaS interfaces use HTTP packages, standard Java APIs, JavaMail API, Java Data Objects (JDO), Java Persistence API (JPA), Python APIs, web integration APIs for different programming languages including, e.g., Rack for Ruby, WSGI for Python, or PSGI for Perl, or other APIs that may be built on REST, HTTP, XML, or other protocols. Clients 102 may access SaaS resources through the use of web-based user interfaces, provided by a web browser (e.g. GOOGLE CHROME, Microsoft INTERNET EXPLORER, or Mozilla Firefox provided by Mozilla Foundation of Mountain View, Calif.). Clients 102 may also access SaaS resources through smartphone or tablet applications, including, e.g., Salesforce Sales Cloud, or Google Drive app. Clients 102 may also access SaaS resources through the client operating system, including, e.g., Windows file system for DROPBOX.

In some embodiments, access to IaaS, PaaS, or SaaS resources may be authenticated. For example, a server or authentication server may authenticate a user via security certificates, HTTPS, or API keys. API keys may include various encryption standards such as, e.g., Advanced Encryption Standard (AES). Data resources may be sent over Transport Layer Security (TLS) or Secure Sockets Layer (SSL).

The client 102 and server 106 may be deployed as and/or executed on any type and form of computing device, e.g. a computer, network device or appliance capable of communicating on any type and form of network and performing the operations described herein. FIGS. 1C and 1D depict block diagrams of a computing device 100 useful for practicing an embodiment of the client 102 or a server 106. As shown in FIGS. 1C and 1D, each computing device 100 includes a central processing unit 121, and a main memory unit 122. As shown in FIG. 1C, a computing device 100 may include a storage device 128, an installation device 116, a network interface 118, an I/O controller 123, display devices 124a-124n, a keyboard 126 and a pointing device 127, e.g. a mouse. The storage device 128 may include, without limitation, an operating system, software, and a software of a genomic data processing system 120. As shown in FIG. 1D, each computing device 100 may also include additional optional elements, e.g. a memory port 103, a bridge 170, one or more input/output devices 130a-130n (generally referred to using reference numeral 130), and a cache memory 140 in communication with the central processing unit 121.

The central processing unit 121 is any logic circuitry that responds to and processes instructions fetched from the main memory unit 122. In many embodiments, the central processing unit 121 is provided by a microprocessor unit, e.g.: those manufactured by Intel Corporation of Mountain View, Calif.; those manufactured by Motorola Corporation of Schaumburg, Ill.; the ARM processor and TEGRA system on a chip (SoC) manufactured by Nvidia of Santa Clara, Calif.; the POWER7 processor, those manufactured by International Business Machines of White Plains, N.Y.; or those manufactured by Advanced Micro Devices of Sunnyvale, Calif. The computing device 100 may be based on any of these processors, or any other processor capable of operating as described herein. The central processing unit 121 may utilize instruction level parallelism, thread level parallelism, different levels of cache, and multi-core processors. A multi-core processor may include two or more processing units on a single computing component. Examples of multi-core processors include the AMD PHENOM IIX2, INTEL CORE i5 and INTEL CORE i7.

Main memory unit 122 may include one or more memory chips capable of storing data and allowing any storage location to be directly accessed by the microprocessor 121. Main memory unit 122 may be volatile and faster than storage 128 memory. Main memory units 122 may be Dynamic random access memory (DRAM) or any variants, including static random access memory (SRAM), Burst SRAM or SynchBurst SRAM (BSRAM), Fast Page Mode DRAM (FPM DRAM), Enhanced DRAM (EDRAIVI), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 122 or the storage 128 may be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 122 may be based on any of the above described memory chips, or any other available memory chips capable of operating as described herein. In the embodiment shown in FIG. 1C, the processor 121 communicates with main memory 122 via a system bus 150 (described in more detail below). FIG. 1D depicts an embodiment of a computing device 100 in which the processor communicates directly with main memory 122 via a memory port 103. For example, in FIG. 1D the main memory 122 may be DRDRAM.

FIG. 1D depicts an embodiment in which the main processor 121 communicates directly with cache memory 140 via a secondary bus, sometimes referred to as a backside bus.

In other embodiments, the main processor 121 communicates with cache memory 140 using the system bus 150. Cache memory 140 typically has a faster response time than main memory 122 and is typically provided by SRAM, BSRAM, or EDRAM. In the embodiment shown in FIG. 1D, the processor 121 communicates with various I/O devices 130 via a local system bus 150. Various buses may be used to connect the central processing unit 121 to any of the I/O devices 130, including a PCI bus, a PCI-X bus, or a PCI-Express bus, or a NuBus. For embodiments in which the I/O device is a video display 124, the processor 121 may use an Advanced Graphics Port (AGP) to communicate with the display 124 or the I/O controller 123 for the display 124. FIG. 1D depicts an embodiment of a computer 100 in which the main processor 121 communicates directly with I/O device 130b or other processors 121′ via HYPERTRANSPORT, RAPIDIO, or INFINIBAND communications technology. FIG. 1D also depicts an embodiment in which local busses and direct communication are mixed: the processor 121 communicates with I/O device 130a using a local interconnect bus while communicating with I/O device 130b directly.

A wide variety of I/O devices 130a-130n may be present in the computing device 100. Input devices may include keyboards, mice, trackpads, trackballs, touchpads, touch mice, multi-touch touchpads and touch mice, microphones, multi-array microphones, drawing tablets, cameras, single-lens reflex camera (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors. Output devices may include video displays, graphical displays, speakers, headphones, inkjet printers, laser printers, and 3D printers.

Devices 130a-130n may include a combination of multiple input or output devices, including, e.g., Microsoft KINECT, Nintendo Wiimote for the WII, Nintendo WII U GAMEPAD, or Apple IPHONE. Some devices 130a-130n allow gesture recognition inputs through combining some of the inputs and outputs. Some devices 130a-130n provides for facial recognition which may be utilized as an input for different purposes including authentication and other commands. Some devices 130a-130n provides for voice recognition and inputs, including, e.g., Microsoft KINECT, SIRI for IPHONE by Apple, Google Now or Google Voice Search.

Additional devices 130a-130n have both input and output capabilities, including, e.g., haptic feedback devices, touchscreen displays, or multi-touch displays. Touchscreen, multi-touch displays, touchpads, touch mice, or other touch sensing devices may use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices may allow two or more contact points with the surface, allowing advanced functionality including, e.g., pinch, spread, rotate, scroll, or other gestures. Some touchscreen devices, including, e.g., Microsoft PIXELSENSE or Multi-Touch Collaboration Wall, may have larger surfaces, such as on a table-top or on a wall, and may also interact with other electronic devices. Some I/O devices 130a-130n, display devices 124a-124n or group of devices may be augment reality devices. The I/O devices may be controlled by an I/O controller 123 as shown in FIG. 1C. The I/O controller may control one or more I/O devices, such as, e.g., a keyboard 126 and a pointing device 127, e.g., a mouse or optical pen. Furthermore, an I/O device may also provide storage and/or an installation medium 116 for the computing device 100. In still other embodiments, the computing device 100 may provide USB connections (not shown) to receive handheld USB storage devices. In further embodiments, an I/O device 130 may be a bridge between the system bus 150 and an external communication bus, e.g. a USB bus, a SCSI bus, a FireWire bus, an Ethernet bus, a Gigabit Ethernet bus, a Fibre Channel bus, or a Thunderbolt bus.

In some embodiments, display devices 124a-124n may be connected to I/O controller 123. Display devices may include, e.g., liquid crystal displays (LCD), thin film transistor LCD (TFT-LCD), blue phase LCD, electronic papers (e-ink) displays, flexile displays, light emitting diode displays (LED), digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays. Examples of 3D displays may use, e.g. stereoscopy, polarization filters, active shutters, or autostereoscopy. Display devices 124a-124n may also be a head-mounted display (HMD). In some embodiments, display devices 124a-124n or the corresponding I/O controllers 123 may be controlled through or have hardware support for OPENGL or DIRECTX API or other graphics libraries.

In some embodiments, the computing device 100 may include or connect to multiple display devices 124a-124n, which each may be of the same or different type and/or form. As such, any of the I/O devices 130a-130n and/or the I/O controller 123 may include any type and/or form of suitable hardware, software, or combination of hardware and software to support, enable or provide for the connection and use of multiple display devices 124a-124n by the computing device 100. For example, the computing device 100 may include any type and/or form of video adapter, video card, driver, and/or library to interface, communicate, connect or otherwise use the display devices 124a-124n. In one embodiment, a video adapter may include multiple connectors to interface to multiple display devices 124a-124n. In other embodiments, the computing device 100 may include multiple video adapters, with each video adapter connected to one or more of the display devices 124a-124n. In some embodiments, any portion of the operating system of the computing device 100 may be configured for using multiple displays 124a-124n. In other embodiments, one or more of the display devices 124a-124n may be provided by one or more other computing devices 100a or 100b connected to the computing device 100, via the network 104. In some embodiments software may be designed and constructed to use another computer's display device as a second display device 124a for the computing device 100. For example, in one embodiment, an Apple iPad may connect to a computing device 100 and use the display of the device 100 as an additional display screen that may be used as an extended desktop. One ordinarily skilled in the art will recognize and appreciate the various ways and embodiments that a computing device 100 may be configured to have multiple display devices 124a-124n.

Referring again to FIG. 1C, the computing device 100 may comprise a storage device 128 (e.g. one or more hard disk drives or redundant arrays of independent disks) for storing an operating system or other related software, and for storing application software programs such as any program related to the software for the genomic data processing system 120. Examples of storage device 128 include, e.g., hard disk drive (HDD); optical drive including CD drive, DVD drive, or BLU-RAY drive; solid-state drive (SSD); USB flash drive; or any other device suitable for storing data. Some storage devices may include multiple volatile and non-volatile memories, including, e.g., solid state hybrid drives that combine hard disks with solid state cache. Some storage device 128 may be non-volatile, mutable, or read-only. Some storage device 128 may be internal and connect to the computing device 100 via a bus 150. Some storage devices 128 may be external and connect to the computing device 100 via an I/O device 130 that provides an external bus. Some storage device 128 may connect to the computing device 100 via the network interface 118 over a network 104, including, e.g., the Remote Disk for MACBOOK AIR by Apple. Some client devices 100 may not require a non-volatile storage device 128 and may be thin clients or zero clients 102. Some storage device 128 may also be used as an installation device 116, and may be suitable for installing software and programs. Additionally, the operating system and the software can be run from a bootable medium, for example, a bootable CD, e.g. KNOPPIX, a bootable CD for GNU/Linux that is available as a GNU/Linux distribution from knoppix.net.

Client device 100 may also install software or application from an application distribution platform. Examples of application distribution platforms include the App Store for iOS provided by Apple, Inc., the Mac App Store provided by Apple, Inc., GOOGLE PLAY for Android OS provided by Google Inc., Chrome Webstore for CHROME OS provided by Google Inc., and Amazon Appstore for Android OS and KINDLE FIRE provided by Amazon.com, Inc. An application distribution platform may facilitate installation of software on a client device 102. An application distribution platform may include a repository of applications on a server 106 or a cloud 108, which the clients 102a-102n may access over a network 104. An application distribution platform may include application developed and provided by various developers. A user of a client device 102 may select, purchase and/or download an application via the application distribution platform.

Furthermore, the computing device 100 may include a network interface 118 to interface to the network 104 through a variety of connections including, but not limited to, standard telephone lines LAN or WAN links (e.g., 802.11, T1, T3, Gigabit Ethernet, Infiniband), broadband connections (e.g., ISDN, Frame Relay, ATM, Gigabit Ethernet, Ethernet-over-SONET, ADSL, VDSL, BPON, GPON, fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMax and direct asynchronous connections). In one embodiment, the computing device 100 communicates with other computing devices 100′ via any type and/or form of gateway or tunneling protocol e.g. Secure Socket Layer (SSL) or Transport Layer Security (TLS), or the Citrix Gateway Protocol manufactured by Citrix Systems, Inc. of Ft. Lauderdale, Fla. The network interface 118 may comprise a built-in network adapter, network interface card, PCMCIA network card, EXPRESSCARD network card, card bus network adapter, wireless network adapter, USB network adapter, modem or any other device suitable for interfacing the computing device 100 to any type of network capable of communication and performing the operations described herein.

A computing device 100 of the sort depicted in FIGS. 1B and 1C may operate under the control of an operating system, which controls scheduling of tasks and access to system resources. The computing device 100 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: WINDOWS 2000, WINDOWS Server 2022, WINDOWS CE, WINDOWS Phone, WINDOWS XP, WINDOWS VISTA, and WINDOWS 7, WINDOWS RT, and WINDOWS 8 all of which are manufactured by Microsoft Corporation of Redmond, Wash.; MAC OS and iOS, manufactured by Apple, Inc. of Cupertino, Calif.; and Linux, a freely-available operating system, e.g. Linux Mint distribution (“distro”) or Ubuntu, distributed by Canonical Ltd. of London, United Kingdom; or Unix or other Unix-like derivative operating systems; and Android, designed by Google, of Mountain View, Calif., among others. Some operating systems, including, e.g., the CHROME OS by Google, may be used on zero clients or thin clients, including, e.g., CHROMEBOOKS.

The computer system 100 can be any workstation, telephone, desktop computer, laptop or notebook computer, netbook, ULTRABOOK, tablet, server, handheld computer, mobile telephone, smartphone or other portable telecommunications device, media playing device, a gaming system, mobile computing device, or any other type and/or form of computing, telecommunications or media device that is capable of communication. The computer system 100 has sufficient processor power and memory capacity to perform the operations described herein. In some embodiments, the computing device 100 may have different processors, operating systems, and input devices consistent with the device. The Samsung GALAXY smartphones, e.g., operate under the control of Android operating system developed by Google, Inc. GALAXY smartphones receive input via a touch interface.

In some embodiments, the computing device 100 is a gaming system. For example, the computer system 100 may comprise a PLAYSTATION 3, or PERSONAL PLAYSTATION PORTABLE (PSP), or a PLAYSTATION VITA device manufactured by the Sony Corporation of Tokyo, Japan, a NINTENDO DS, NINTENDO 3DS, NINTENDO WII, or a NINTENDO WII U device manufactured by Nintendo Co., Ltd., of Kyoto, Japan, an XBOX 360 device manufactured by the Microsoft Corporation of Redmond, Wash.

In some embodiments, the computing device 100 is a digital audio player such as the Apple IPOD, IPOD Touch, and IPOD NANO lines of devices, manufactured by Apple Computer of Cupertino, Calif. Some digital audio players may have other functionality, including, e.g., a gaming system or any functionality made available by an application from a digital application distribution platform. For example, the IPOD Touch may access the Apple App Store. In some embodiments, the computing device 100 is a portable media player or digital audio player supporting file formats including, but not limited to, MP3, WAV, M4A/AAC, WMA Protected AAC, AIFF, Audible audiobook, Apple Lossless audio file formats and .mov, .m4v, and .mp4 MPEG-4 (H.264/MPEG-4 AVC) video file formats.

In some embodiments, the computing device 100 is a tablet e.g. the IPAD line of devices by Apple; GALAXY TAB family of devices by Samsung; or KINDLE FIRE, by Amazon.com, Inc. of Seattle, Wash. In other embodiments, the computing device 100 is an eBook reader, e.g. the KINDLE family of devices by Amazon.com, or NOOK family of devices by Barnes & Noble, Inc. of New York City, N.Y.

In some embodiments, the communications device 102 includes a combination of devices, e.g. a smartphone combined with a digital audio player or portable media player. For example, one of these embodiments is a smartphone, e.g. the IPHONE family of smartphones manufactured by Apple, Inc.; a Samsung GALAXY family of smartphones manufactured by Samsung, Inc.; or a Motorola DROID family of smartphones. In yet another embodiment, the communications device 102 is a laptop or desktop computer equipped with a web browser and a microphone and speaker system, e.g. a telephony headset. In these embodiments, the communications devices 102 are web-enabled and can receive and initiate phone calls. In some embodiments, a laptop or desktop computer is also equipped with a webcam or other video capture device that enables video chat and video call.

In some embodiments, the status of one or more machines 102, 106 in the network 104 are monitored, generally as part of network management. In one of these embodiments, the status of a machine may include an identification of load information (e.g., the number of processes on the machine, CPU and memory utilization), of port information (e.g., the number of available communication ports and the port addresses), or of session status (e.g., the duration and type of processes, and whether a process is active or idle). In another of these embodiments, this information may be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

B. Computer Implemented Method for Identifying Forward and Reverse Primers from Genomic Data

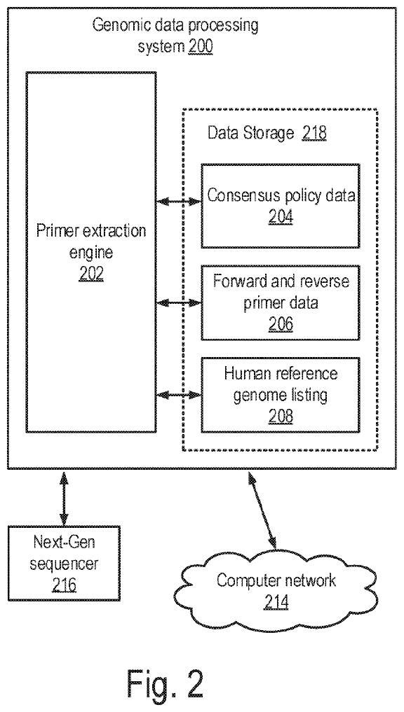

FIG. 2 illustrates a genomic data processing system 200, similar to the genomic data processing system 120 shown in FIG. 1C. In particular, the genomic data processing system 200 processes genomic data to determine forward and reverse primers used for generating the genomic data. Selection of appropriate primers is important because primers that lack the appropriate degree of sequence complementarity can result in the production of sequence reads that are not representative of the relevant V-J segments, and may consequently reduce the computational accuracy of various parameters such as sequence read frequencies for a particular V-J clone. As primers used for generating V-J sequence reads received from some next-generation sequencers are not known, processing the received sequence reads may result in reduced accuracy. By identifying the primers from the sequence reads, appropriate primers can be selected for further analysis to improve accuracy. Furthermore, by knowing the identity of the primers used to process the samples, a more accurate analysis of the clonality of the samples can be performed as described herein.

The genomic data processing system 200 includes a primer extraction engine 202 and data storage 218. The data storage 218 can include consensus policy data 204, forward and reverse primer data 206, and human reference genome listing 208. The genomic data processing system 200 can be coupled to a computer network 214, which can include one or more wired or wireless networks such as, for example, Ethernet, Internet, WiFi network, Bluetooth network, and the like. The genomic data processing system 200 can be implemented using the computing systems discussed above in relation to FIGS. 1A-1D.

The genomic data processing system 200 can receive data from a next-generation genomic sequencer (“NG sequencer”) 216, such as, for example, an Illumina sequencer, a Lymphotrac sequencer, an Ion Torrent sequencer, and a 454 pyro-sequencer. The NG sequencer 216 can provide detailed chromosome analysis, and can employ techniques such as array comparative genomic hybridization (CGH), microarray, oligo array, single nucleotide polymorphism (SNP) array, whole genome array (WGA), and the like. The NG sequencer 216 can provide raw genomic data to the genomic data translation system 200. In particular, the NG sequencer 216 can provide genomic data derived from biological samples that have been processed with forward and reverse primers in a next generation sequencing assay.

During development, the antigen receptor genes in lymphoid cells undergo somatic gene rearrangement. For example, during B-cell development, genes encoding the IGH molecules are assembled from multiple gene segments that undergo rearrangements and selection. These gene rearrangements of the V, D, and J generate V-D-J combinations of unique length and sequence for each cell. For example, the immunoglobulin heavy chain (IGH) gene locus on chromosome 14 (14q32.3) includes 46-52 functional and 30 non-functional variable (V) gene segments, 27 functional diversity (D) gene segments, and 6 functional joining (J) gene segments spread over 1250 kilobases.

Since leukemias and lymphomas originate from the malignant transformation of individual lymphoid cells, all leukemias and lymphomas generally share one or more cell-specific or “clonal” antigen receptor gene rearrangements. Tests that detect IGH clonal rearrangements can be useful in the study of B cell malignancies.

PCR-based assays identify clonality on the basis of over-representation of amplified V-D-J (or incomplete D-J products) gene rearrangements following their separation using gel electrophoresis. Though sensitive and suitable for testing small amounts of DNA, these assays cannot readily differentiate between clonal populations and multiple rearrangements that might lie beneath a single-sized peak, and are not designed to identify the specific V-J DNA sequence that is required to track subsequent analyses.

PCR assays are routinely used for the identification of clonal B- and T-cell populations. These assays amplify the DNA between primers that target the conserved framework of the V regions and the conserved J regions of antigen receptor genes. These conserved regions, where primers target, lie on either side of an area where programmed genetic rearrangements occur during the maturation of all B and T lymphocytes. It is a result of these genetic rearrangements that different populations of the B and T lymphocytes arise.

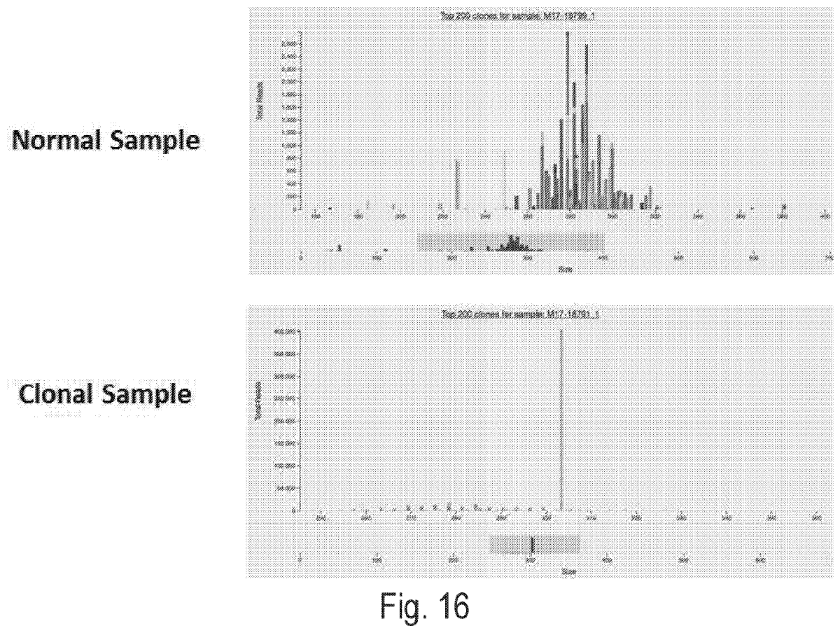

The antigen receptor genes that undergo rearrangements are the immunoglobulin heavy chain (IGH) and light chain loci (IGK and IGL) in B cells, and the T-cell receptor gene loci (TRA, TRB, TRG, and TRD) in T cells. Each B and T cell has one or two productive V-J rearrangements that are unique in both length and sequence. Therefore, when DNA from a normal or polyclonal population is amplified using DNA primers that flank the V-J region, amplicons that are unique in both sequence and length, reflecting the heterogeneous population, are generated. See FIG. 16. For samples containing clonal populations, the yield is one or two prominent amplified products of the same length and sequence that are detected with significant frequency of occurrence, within a diminished polyclonal background amplified at a lower frequency. See FIG. 16.

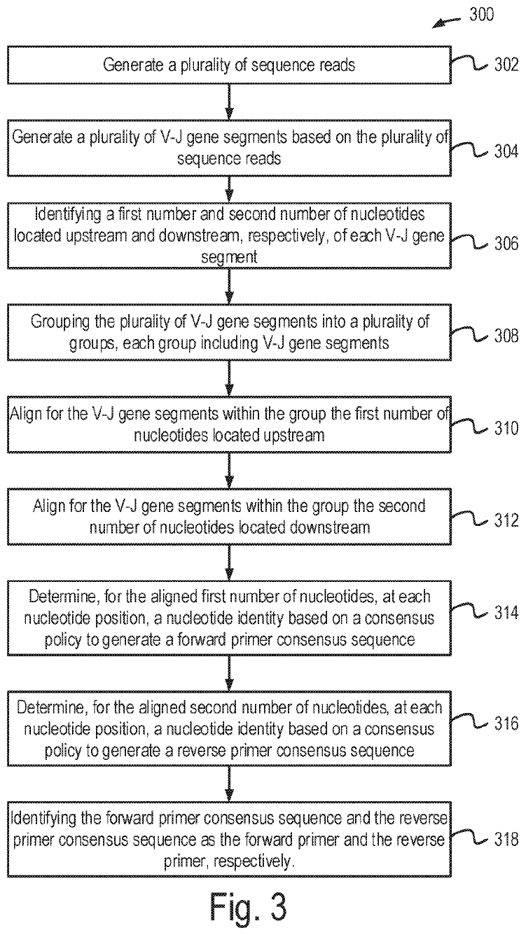

FIG. 3 illustrates a flow diagram of a primer extraction process 300. The process 300 includes generating a plurality of sequence reads (block 302). The process 300 can be executed, for example, by the primer extraction engine 202 shown in FIG. 2. The primer extraction engine 202 can receive genomic data from the NG sequencer 216. The genomic data, as mentioned above, can include genomic data derived from biological samples that have been processed with forward and reverse primers in a next generation sequencing assay. In particular, the genomic data can include a number of sequence reads resulting from the use of forward and reverse primers. The sequence may include the sequence of nucleotides that have been trimmed of any information related to the forward and reverse primers used to generate the sequence read.

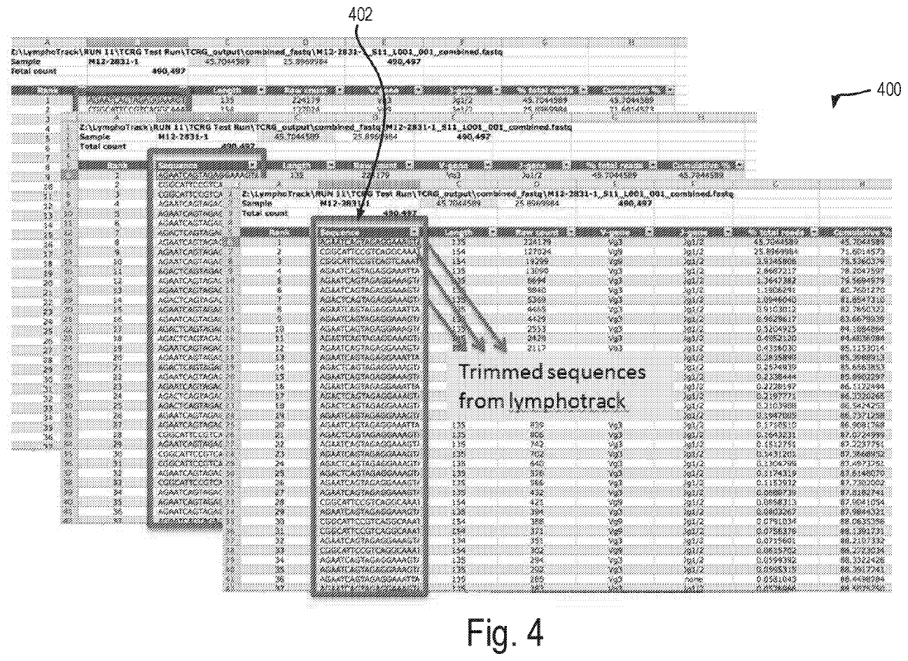

FIG. 4 illustrates screenshots 400 of generating example sequence reads from genomic data provided by an example next generation sequencer. In particular, the screenshots 400 illustrate an output of a Lymphotrack® Data Analysis Tool, which is a bioinformatics data analysis tool that is used for detecting V-J clone sequences within the next-generation sequencing (NGS) output from a LymphoTrack Assay. The output includes a column of sequence reads 402, which have been trimmed to exclude any forward and reverse primer information. The output further includes the raw count, length, and frequency (% total reads) of each detected V-J clone sequence. The primer extraction engine 202 receives these sequence reads 402 (and other output data) from the NG sequencer 216 for further processing. In some implementations, the primer extraction engine 202 can generate sequence reads data structures for each of the sequence reads 402 and store the sequence reads data structures in memory. The data structure can include the sequence read, and the additional output data provided by the NG sequencer 216.

Referring again to FIG. 3, the process 300 includes generating a plurality of V-J gene segments (block 304). The primer extraction engine 202 can lookup each sequence read received from the NG sequencer 212 in a human reference genome listing 208 to determine a corresponding V-J segment. The human reference genome listing can include human reference genome data or various builds such as hg16, hg17, hg18, hg19, and hg38.

The process 300 includes identifying a first number and second number of nucleotides located upstream and downstream, respectively, of each V-J gene segment (block 306). In particular, the primer extraction engine 202 can compare each V-J gene segment with the genomic data received from the NG sequencer 212 to identify for the corresponding V-J segment a first number of nucleotides located upstream of the corresponding V-J gene segment and a second number of nucleotides located downstream of the corresponding gene segment.

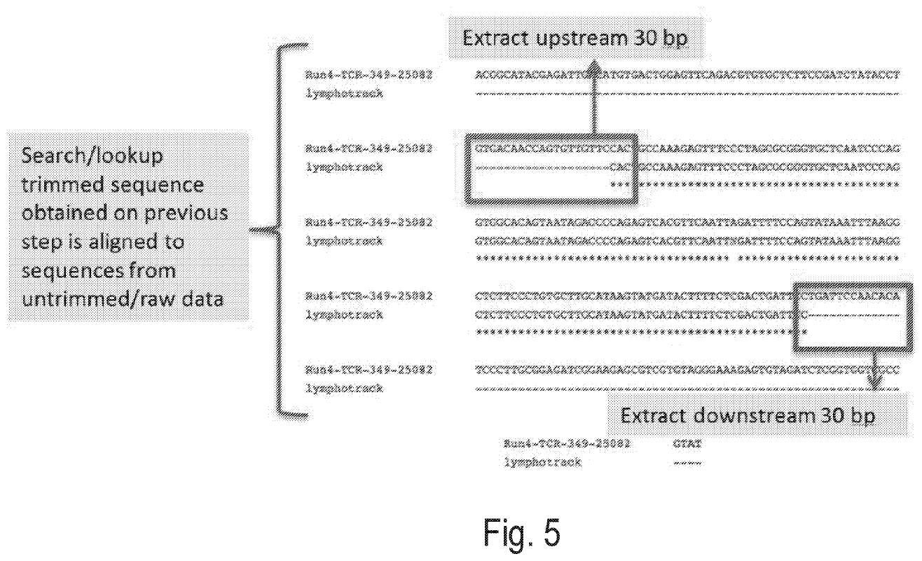

FIG. 5 shows one example of identifying a first number and second number of nucleotides located upstream and downstream, respectively, of each V-J gene segment. In particular, FIG. 5 shows the primer extraction engine 202 comparing the V-J gene segment generated from the Lymphotrac genomic data with the genomic data (labeled “Run4-TCR-349-25082”) received from the NG sequencer 212 to extracting 30 base pairs upstream and 30 base pairs downstream of the V-J gene segment. In some implementations, the number of base pairs upstream and downstream can be different from the 30 shown in FIG. 5. For example, the primer extraction engine 202 can instead extract about 20 to about 35 or about 25 base pairs upstream and downstream of the V-J gene segment.

In some embodiments of the methods disclosed herein, the first number of nucleotides located upstream of the corresponding V-J gene segment may be between 20-30 base pairs in length and may further comprise a next-generation sequencing (NGS)-compatible adapter sequence. Additionally or alternatively, in some embodiments of the methods disclosed herein, the second number of nucleotides located downstream of the corresponding V-J gene segment may be between 20-30 base pairs in length and may further comprise a NGS-compatible adapter sequence and/or a patient specific barcode sequence (also known as an index tag, or a multiplex identifier (MID)). Examples of NGS-compatible adapter sequences include a P5 adapter, P7 adapter, P1 adapter, A adapter, or Ion Xpress™ barcode adapter. Other adapter sequences are known in the art. Some manufacturers recommend specific adapter sequences for use with the particular sequencing technology and machinery that they offer. In some implementations, the first number can be 20 base pairs in length. In some implementations, the first number can be 30 base pairs in length. In some implementations, the first number can be between 5-100, 10-90, 10-80, 10-70, 10-60, 10-50, 10-40, or 10-30 base pairs in length. In some implementations, the first number can be greater than 100 base pairs in length. In some implementations, the second number can be 20 base pairs in length. In some implementations, the second number can be 30 base pairs in length. In some implementations, the second number can be between 5-100, 10-90, 10-80, 10-70, 10-60, 10-50, 10-40, or 10-30 base pairs in length. In some implementations, the second number can be greater than 100 base pairs in length.

In some embodiments, the first number of nucleotides located upstream of the V-J gene segments within each group contain the same adapter sequence. Additionally or alternatively, in some embodiments, the second number of nucleotides located downstream of the V-J gene segments within each group contain the same adapter sequence.

In some embodiments, the second number of nucleotides located downstream of the corresponding V-J gene segment comprise an adapter sequence that is distinct from the adapter sequence present in the first number of nucleotides located upstream of the corresponding V-J gene segment.

In some embodiments of the methods disclosed herein, the second number of nucleotides located downstream of the corresponding V-J gene segment and/or the first number of nucleotides located upstream of the corresponding V-J gene segment contain an adapter sequence that further comprises an identical index sequence or barcode sequence that indicates the patient from which the sample was obtained. For example, the barcode sequence for all samples obtained from a single patient may be different from the barcode sequences of the samples obtained from different patients. As such, the use of barcode sequences permits multiple samples from different patients to be pooled per sequencing run and the sample source subsequently ascertained based on the index sequence. In some embodiments, samples derived from up to 48 separate patients are pooled prior to sequencing

Referring again to FIG. 3, the process 300 includes grouping the plurality of V-J gene segments into a plurality of groups, each group including V-J gene segments (block 308). In particular, the prime extraction engine 202 can group the plurality of V-J gene segments into a plurality of groups. Each group of the plurality of groups can include V-J gene segments having a same V-J identity.

The process 300 includes the primer extraction engine 202 performing actions in each of the following blocks 310-318 for each group of V-J gene segments from the plurality of groups. In particular, the primer extraction engine 202, for all V-J segments in the group, can align the first number of nucleotides located upstream of the V-J gene segments (block 310) and, for all V-J segments in the group, align the second number of nucleotides located downstream of the V-J gene segments (block 312)

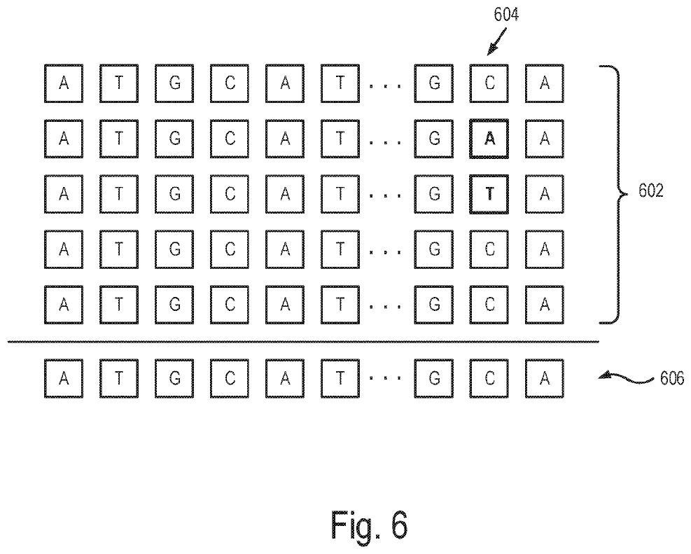

FIG. 6 illustrates an alignment of the first number of nucleotides 602 associated with V-J gene segments within a group. For example, the primer extraction engine 202 can store the first number of nucleotides for each V-J gene segment within a group in an array data structure, with each position in one dimension of the array corresponding to a position of the nucleotide. While only five first number of nucleotides are shown in FIG. 6, this is only an example for ease of illustration, and that the primer extraction engine 202 can align as many first number of nucleotides as the V-J segments in the group. The primer extraction engine 202 can similarly align the second number of nucleotides associated with V-J gene segments within the group.

The process 300 includes determining for the aligned first number of nucleotides, at each nucleotide position, a nucleotide identity based on a consensus policy to generate a forward primer consensus sequence (block 314). In particular, the primer extraction engine 202 can determine the level of agreement in the identity of a nucleotide for each position of the first number of nucleotides associated with the V-J gene segments within the group. FIG. 6 shows a forward primer consensus sequence 606 determined by the primer extraction engine 202 based on the first number of nucleotides 502 and the consensus policy data 204 (FIG. 2). As shown in FIG. 6, the nucleotide identities of all the positions except position 604 are identical. In one example, the consensus policy can indicate that if the nucleotide identities at a position do not match, then the nucleotide having more than 50% proportion of all the nucleotides at that position can be selected to be the consensus nucleotide identity. The primer extraction engine 202 can determine that at position 604, the nucleotide identities do not match, as the second and the third nucleotide are “A” and “T” while the other nucleotides are “C”. The primer extraction engine 202 can then determine the proportion of each identity at position 604. Thus, the primer extraction engine 202 can determine that the identity “C” occurs three times, while the identities “A” and “T” each occur once. The proportion of the identity “C” is 60%, while that of each of the identities “A” and “T” is 20%. The primer extraction engine 202, based on the consensus policy, can then select the identity “C” as the consensus identity for position 604. Other consensus policies can also be used. Such as for example, the consensus identity being the identity that has the greatest occurrence at the position 604, or the identity occurring greater than a predetermined threshold value, etc. In some implementations, the percentage proportion discussed above can range from about 20% to about 80% or about 30% to about 70%, or about 40% to about 60% or at least 50%. In some implementations, the primer extraction engine 202, in the absence of the any identity satisfying the consensus policy can include a “wild card identity” at that location. In some other implementations, the primer extraction engine 202 can modify the consensus policy such that a consensus identity can be determined. For example, the extraction engine 202 can change the % threshold value until a single identity can be determined for that position.

The process 300 can include determining, for the aligned second number of nucleotides, at each nucleotide position, a nucleotide identity based on a consensus policy to generate a reverse primer consensus sequence (block 316). The primer extraction engine 202 can determine the reverse primer consensus sequence in a manner similar to that discussed above in relation to determining the forward primer consensus sequence.

The process 300 can include identifying the forward primer consensus sequence and the reverse primer consensus sequence as the forward primer and the reverse primer, respectively (block 318). The primer extraction engine 202 can store the forward and reverse primer consensus sequences for each group as forward and reverse primer sequence data 206. The primer extraction engine 202 can identify the determined forward and reverse consensus primer sequences as the forward and reverse primer sequences used by the NG sequencer 212 to generate the sequence reads.

The process 300 may also include the primer extraction engine 202 generating additional forward and reverse primers from additional biological samples, and storing the detected forward and reverse primers in the forward and reverse primer data 206. Thus, the primer extraction engine 202 can build a library of forward and reverse primers that can be used to generate sequence reads, which in turn can be used to detect clonality at higher accuracy.

C. Computer Implemented Method for Detecting Clonality in Genomic Data

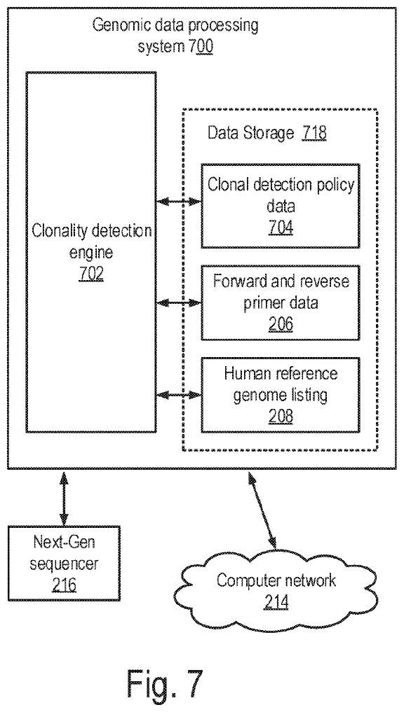

FIG. 7 illustrates a genomic data processing system 700, similar to the genomic data processing system 120 shown in FIG. 1C. In particular, the genomic data processing system 700 processes genomic data to detect clonal V-J segments in the genomic data. The genomic data processing system 700 includes a clonal detection engine 702 and data storage 718. The data storage 718 can include clonal detection policy data 704, forward and reverse primer data 206, and human reference genome listing 208. The forward and reverse primer data 206 can include the forward and reverse primers extracted using the process 300 discussed above in relation to FIGS. 2-6. The genomic data processing system 700 can be coupled to a computer network 214, which can include one or more wired or wireless networks such as, for example, Ethernet, Internet, WiFi network, Bluetooth network, and the like. The genomic data processing system 700 can be implemented using the computing systems discussed above in relation to FIGS. 1A-1D.

The genomic data processing system 700 can receive data from the NG sequencer 216, such as, for example, an Illumina sequencer, a Lymphotrac sequencer, an Ion Torrent sequencer, and a 454 pyro-sequencer. The NG sequencer 216 can provide detailed chromosome analysis, and can employ techniques such as array comparative genomic hybridization (CGH), microarray, oligo array, single nucleotide polymorphism (SNP) array, whole genome array (WGA), and the like. The NG sequencer 216 can provide raw genomic data to the genomic data translation system 200. In particular, the NG sequencer 216 can provide genomic data derived from biological samples that have been processed with forward and reverse primers in a next generation sequencing assay. In some embodiments, the biological samples are derived from the same patient. In other embodiments, the biological samples are derived from the different patients. In some implementations, the genome data processing system 700 can provide the NG sequencer 216 with the forward and reverse primers included in the forward and reverse primer data 206, and receive genomic data from the NG sequencer 216 that has been derived from biological samples that have been processed using the same forward and reverse primers.

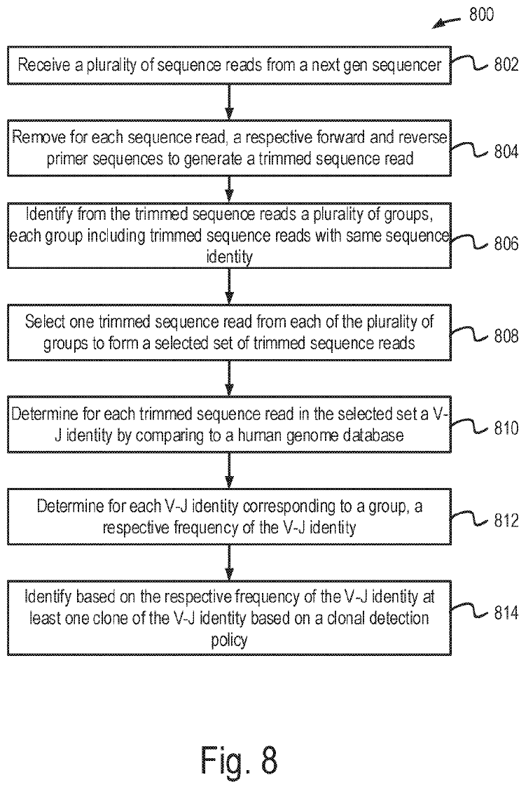

FIG. 8 illustrates a flow diagram of a clonal detection process 800. The process 800 includes receiving a plurality of sequence reads from a next gen sequencer (block 802). In particular, the clonal detection engine 702 can receive, from the NG sequencer 216, a plurality of sequence reads associated with a sample obtained from a subject. Each of the plurality of sequence reads can represent at least one of coding gene segments and non-coding gene segments. The sequence reads received by the clonal detection engine 702 can be determined based on the forward and reverse primer data 206. That is, the sequence reads can be based on the primers determined using the process 300 discussed above in relation to FIGS. 2-6.

The process 800 can include removing, for each sequence read, a respective forward and reverse primer sequences to generate a trimmed sequence read (block 804). In particular, the clonal detection engine 702 can remove for each sequence read in the plurality of sequence reads a respective forward primer sequence and a respective reverse primer sequence to generate a corresponding trimmed sequence read.

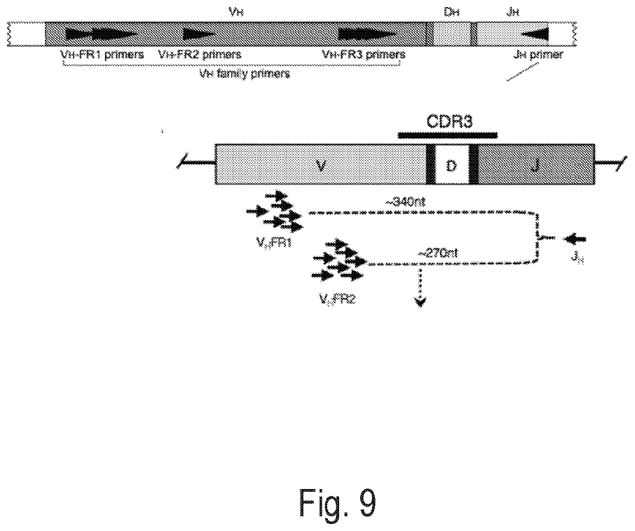

FIG. 9 shows an example representation of forward and reverse primers for a plurality of sequence reads. In particular, FIG. 9 shows the V-D-J regions of the IGH gene. The arrows represent exemplary sites of forward primers binding within the FR1, FR2, and FR3 regions of the V gene segment and the reverse primers binding with the JH region of the J gene segment. The forward and reverse primers identified above can then be removed from the sequence reads to generate corresponding trimmed sequence reads.

Referring again to FIG. 8, the process 800 can include identifying from the trimmed sequence reads a plurality of groups, each group including trimmed sequence reads with same sequence identity (block 806). In particular, the clonal detection engine 702 can identify from the trimmed sequence reads generated from the plurality of sequence reads, a plurality of groups of trimmed sequence reads, where each group includes trimmed sequence reads having a same sequence identity. In some implementations, the same sequence identity can be determined from comparing the trimmed sequence reads to each other, and determining a sequence of nucleotides that are common in the compared trimmed sequence reads. By repeatedly comparing the trimmed sequence reads to each other, groups of trimmed sequence reads can be determined, where each trimmed sequence read in a group includes the same sequence identity, or a common nucleotide sequence.

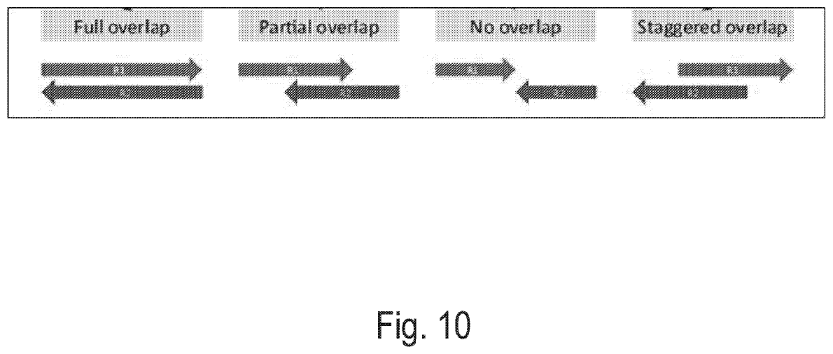

FIG. 10 shows an example representation of identifying a plurality of groups of trimmed sequence reads. The clonal detection engine 702 compares two distinct trimmed sequence reads. The two trimmed sequence reads may completely or incompletely (partial or staggered) overlap with each other or not overlap at all. Overlapping (full, partial, or staggered) trimmed sequence reads indicate that the two trimmed sequence reads include the same sequence identity, and should be grouped together in the same group. In some embodiments, the non-overlapping trimmed sequence reads may not be grouped together in the same group.

Referring again to FIG. 8, the process 800 can include selecting one trimmed sequence read from each of the plurality of groups to form a selected set of trimmed sequence reads (block 808). In particular, the clonal detection engine 702 can select a representative trimmed sequence read from the plurality of trimmed sequence reads in the same group. The clonal detection engine can similarly select representative trimmed sequence reads from all the groups. The clonal detection engine 702 can form a set selected set of trimmed sequence reads that include all the selected representative trimmed sequence reads.

The process 800 can include determining for each trimmed sequence read in the selected set a V-J identity by comparing to a human genome database (block 810). In particular, the clonal detection engine 702 can compare each trimmed sequence read in the selected set of trimmed sequence reads to the human reference genome listing 208 (FIG. 7) that includes associations between nucleotide sequences and V-J identities to determine a corresponding V-J identity.

The process 800 can include determining for each V-J identity corresponding to a group, a respective frequency of the V-J identity (block 812). In particular, the clonal detection engine 202 can determine for each V-J identity corresponding to a group of the plurality of groups of trimmed sequence reads, a respective frequency of the V-J identity based on a number of trimmed sequence reads included in the group. The clonal detection engine 702 can maintain a count of the number of trimmed sequence reads within each group, and identify this number as a frequency of the V-J identity associated with the group.

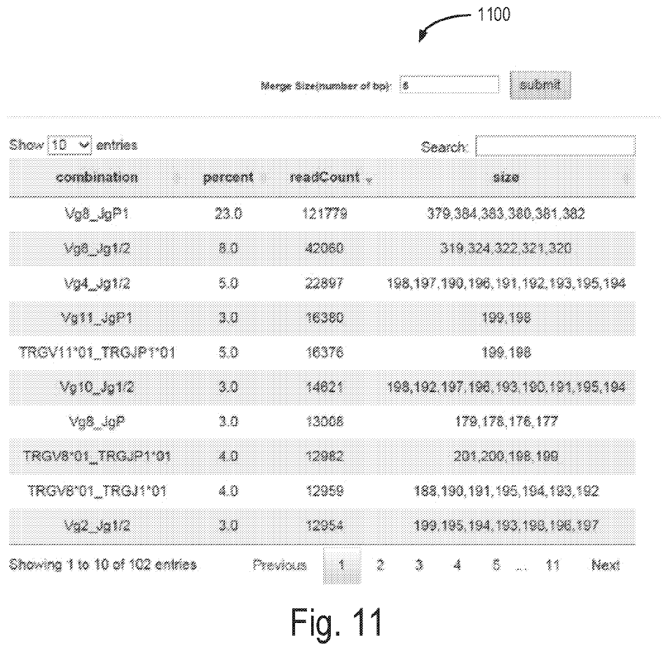

FIG. 11 shows an example output 1100 generated by the clonal detection engine 702. In particular, the clonal detection engine 702 can generate the output 1100 that shows frequency of V-J identities (in relation to other V-J identities). The “combination” column includes V-J identities, and the “percent” column indicates the frequency of the identity as a proportion of sum of the frequencies of all the V-J identities.

The process 800 can include identifying based on the respective frequency of the V-J identity at least one clone of the V-J identity based on a clonal detection policy (block 814). In particular, the clonal detection engine 702 can identify, based on the respective frequency of the V-J identity corresponding to a first group of the plurality of groups of trimmed sequence reads, at least one clone of the V-J identity based on a clonal detection policy.

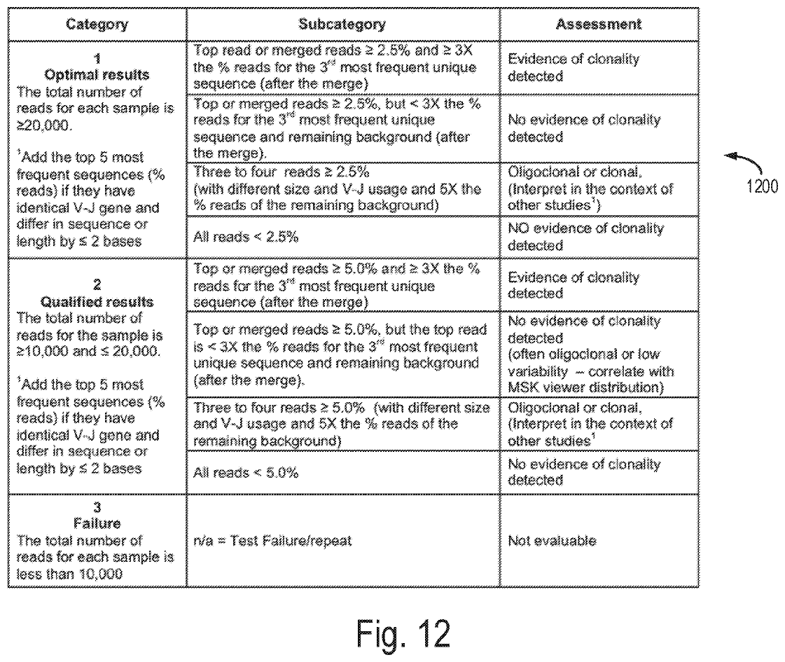

FIG. 12 illustrates a set of clonal detection policies 1200. The detection policies can be stored in the clonal detection policy data 704 (FIG. 7) of the genomic data processing system 700. The clonal detection policies can include three categories of rules: category 1: optimal category, category 2: qualified results, and category 3: Failure. Each category can include a sub-category or rules and corresponding assessments. The various assessments can include “evidence of clonalality detected,” “no evidence of clonality detected,” oligoclonal or clonal,” and “not evaluable.” The assessments can further include suggestions for interpreting the data using other studies or data.

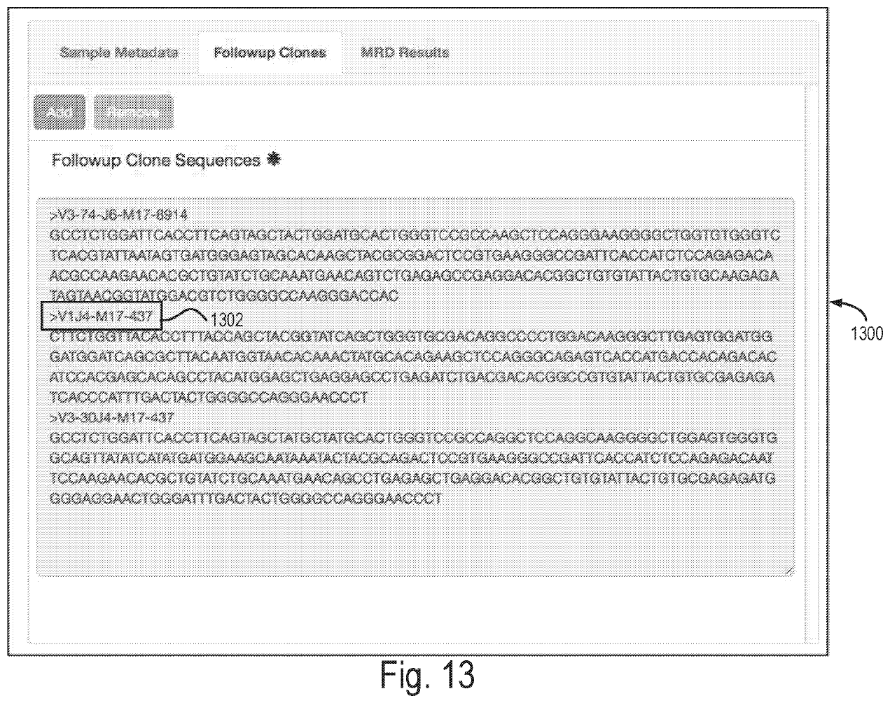

FIG. 13 illustrates follow-up data 1300 related to clone follow-up process. In some implementations, the genomic data processing system 700 can be used to generate V-J identities of the same patient at a different time, such, for example, after a particular treatment. The V-J identities, and the corresponding frequencies, determined in the follow-up data can be stored in memory and compared with the V-J identities and frequencies generated in the past for the same patient. In some implementations, clone sequences identified in a particular patient sample are stored in memory. After a follow-up NGS assay in the same patient sample, the previously identified clone sequences for the patient sample are retrieved, and are queried within the new follow-up sample from the patient. The results are summarized and saved in a database, which can then be made available through a user interface. For example, as shown in FIG. 13, a V-J identity 1302 can be stored in memory and compared with V-J identities already stored in memory.

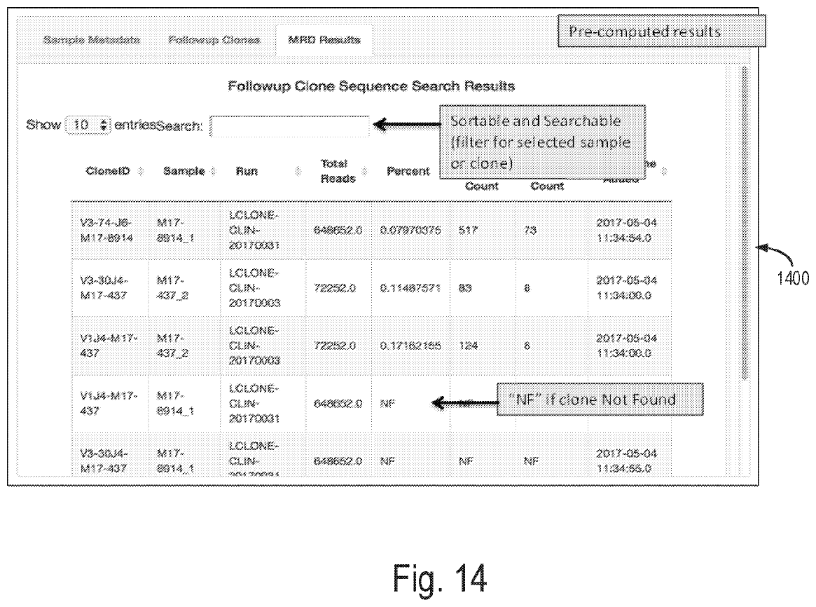

FIG. 14 illustrates a user interface for displaying the clones associated with a patient after a clone follow-up process. In particular, FIG. 14 shows how the results of the follow-up assay from the same sample can be readily accessed by querying for the patient sample or a particular V-J clone. The V-J clone 1302 shown in FIG. 13 is indicated as not found (NF) in the follow-up process.

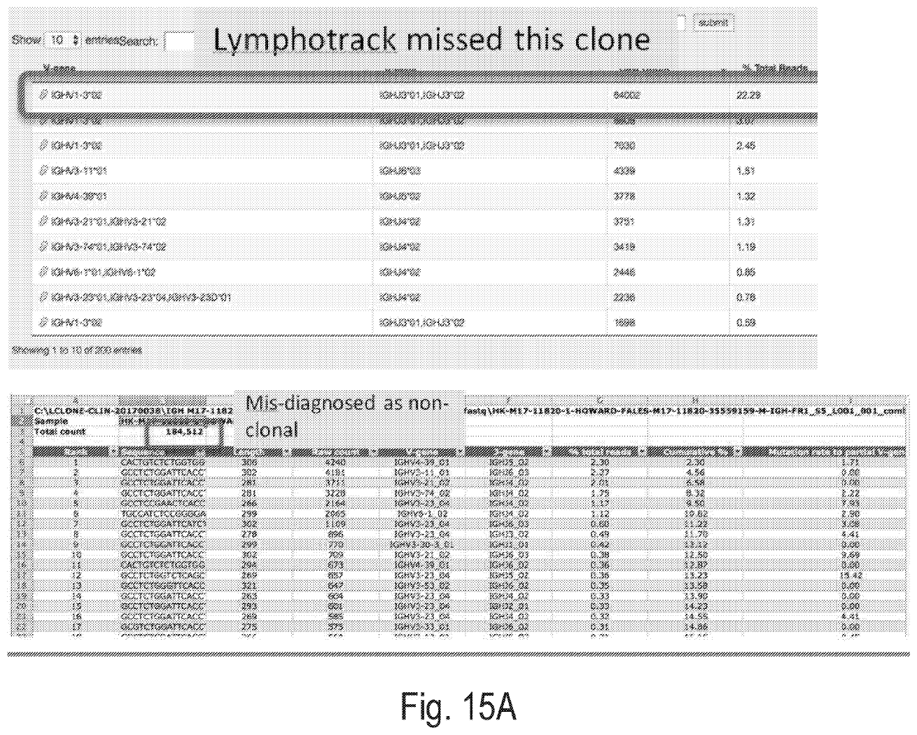

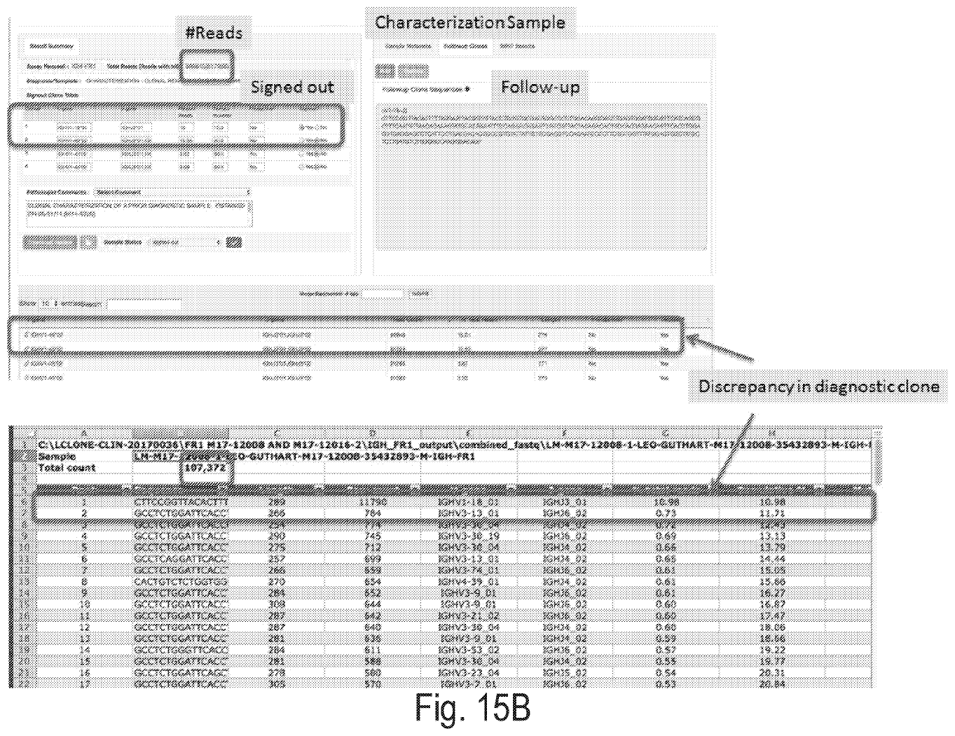

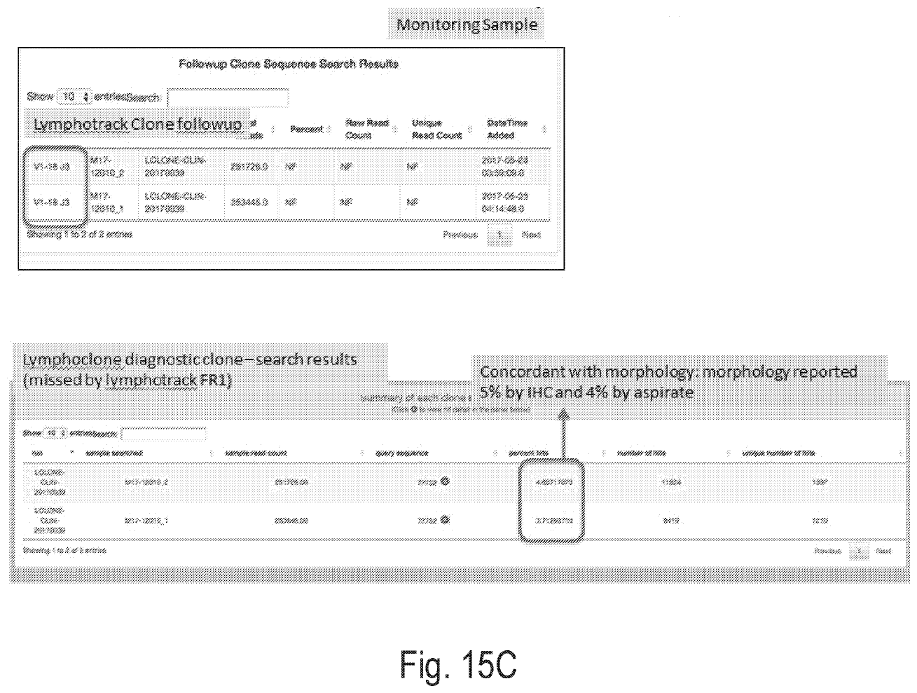

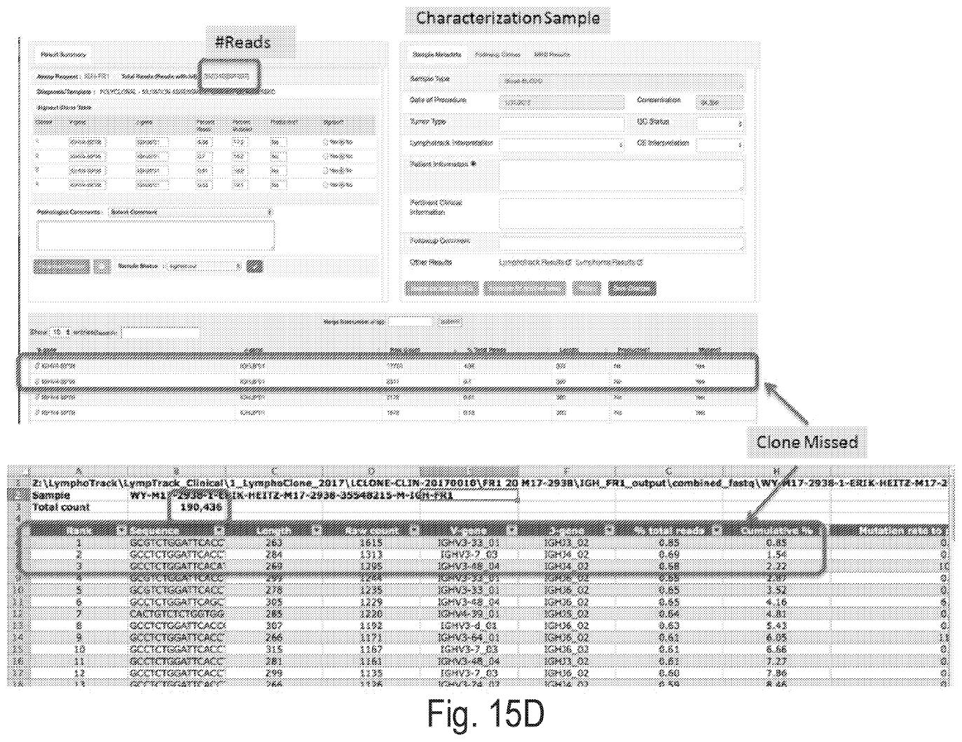

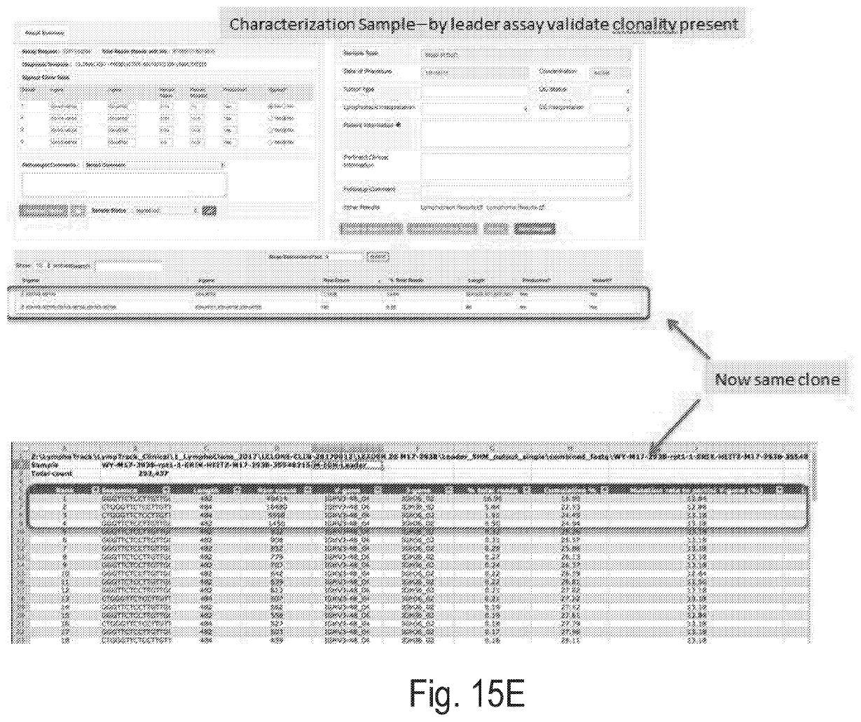

FIGS. 15A-15E show a comparison between the clonal detection results achieved using the conventional Lymphotrack® Data Analysis Tool versus the clonal detection methods of the present technology. FIG. 15A demonstrates that the clonal detection methods disclosed herein were successful in identifying the presence of a dominant V-J clone (V1-3-J3) in a patient sample that was not detected when the conventional Lymphotrack® Data Analysis Tool was used to analyze the same patient sample. The patient sample was subjected to a IGH FR1 assay. FIG. 15B demonstrates that the clonal detection methods disclosed herein were successful in identifying the presence of a dominant V-J clone (V1-45-J3) in a patient sample that was not detected when the conventional Lymphotrack® Data Analysis Tool was used to analyze the same patient sample. The patient sample was subjected to a IGH FR1 assay. FIG. 15C demonstrates that the clonal detection methods disclosed herein are useful for detecting the loss of a previously identified V-J clone (V1-18-J3) in a patient sample during a follow-up NGS-assay. This apparent loss of the V-J clone (V1-18-J3) was not detected when the conventional Lymphotrack® Data Analysis Tool was used to analyze the same patient sample during the follow-up NGS-assay. The patient sample was subjected to a IGH FR1 assay. FIG. 15D demonstrates that the clonal detection methods disclosed herein were successful in identifying the presence of a dominant V-J clone (V4-59-J6) in a patient sample that was not detected when the conventional Lymphotrack® Data Analysis Tool was used to analyze the same patient sample. The patient sample was subjected to a IGH FR1 assay. FIG. 15E shows that both conventional Lymphotrack® Data Analysis Tool and the clonal detection methods disclosed herein identified the same dominant V-J clone when the patient sample described in FIG. 15D was subjected to IGHV leader somatic hypermutation assay.

FIG. 15A demonstrate that the clonal detection methods of the present technology are capable of detecting clonal events in a patient sample that were not detectable when the conventional Lymphotrack® Data Analysis Tool was used to analyze the same patient samples. The superior performance of the methods disclosed herein is attributable at least in part to the primer trimming step (as determined by the consensus policies described herein to generate reverse primer consensus sequences and forward primer consensus sequences for the various V-J segments) and the merge read step described in FIG. 11. As shown in FIGS. 15A and 15D, both patient samples were subjected to a IGH FR1 assay, and then processed using the conventional Lymphotrack® Data Analysis Tool as well the clonal detection process discussed above in relation to FIG. 8.

FIG. 15A demonstrates that the conventional Lymphotrack® Data Analysis Tool failed to detect the presence of a dominant V-J clone (V1-343) in a patient sample. In contrast, the clonal detection methods of the present technology successfully detected the presence of the dominant V1-3-J3 clone in the same patient sample. The accuracy of these results was independently confirmed using secondary assays such as capillary electrophoresis and IGHV leader somatic hypermutation assay, which confirmed the presence of the dominant V1-3 clone in the patient sample. These results are significant because the patient sample would have been erroneously characterized as “non-clonal” because the conventional Lymphotrack® Data Analysis Tool failed to detect the dominant V1-3-J3 clone in the patient sample.

Likewise, FIG. 15D demonstrates that the conventional Lymphotrack® Data Analysis Tool failed to detect the presence of a dominant V-J clone (V4-59-J6) in the patient sample. In contrast, the clonal detection methods of the present technology successfully detected the presence of the dominant V4-59-J6 in the same patient sample. These results are significant because the patient sample would have been erroneously characterized as “non-clonal” if one were to solely rely on the IGH FR1 assay results that were generated using the conventional Lymphotrack® Data Analysis Tool. In contrast, FIG. 15E which shows the IGHV leader somatic hypermutation assay results on the same patient sample confirm that the patient sample was actually a clonal sample (identified as clonal using both the conventional Lymphotrack® Data Analysis Tool demonstrate and the clonal detection methods described herein).

Similarly, FIG. 15B demonstrates that the clonal detection methods disclosed herein were successful in identifying the presence of a dominant V1-45-J3 clone in a patient sample that was not detected when the conventional Lymphotrack® Data Analysis Tool was used to analyze the same patient sample.

FIG. 15B shows that the dominant V1-18-J3 clone was initially detected in a patient sample using either the conventional Lymphotrack® Data Analysis Tool or the clonal detection methods described herein. However, as shown in FIG. 15C, the clonal detection methods disclosed herein were capable of detecting the loss of the V1-18-J3 clone in the same patient sample during a follow-up NGS-assay. This apparent loss of the V1-18-J3 clone was not observed when the conventional Lymphotrack® Data Analysis Tool was used to analyze the same patient sample during the follow-up NGS-assay. The reduced frequency of the V1-18-J3 clone was independently confirmed using secondary morphological assays such as immunohistochemistry (IHC).

Additionally or alternatively, in some embodiments, the at least one clonal V-J gene segment in the sample further comprises a Diversity (D) region. The sample may be a DNA or RNA sample and can optionally be derived from T lymphocytes or B lymphocytes. Examples of T lymphocytes include CD4+ helper T cells, CD8+ cytotoxic T cells, memory T cells, gamma-delta T cells, and regulatory T cells. Examples of B lymphocytes include consisting of plasma cells, memory B cells, follicular B cells, marginal zone B cells, and regulatory B cells.

Additionally or alternatively, in some embodiments, the sample is obtained from a patient that is diagnosed with, is suspected of having, or is at risk for a lymphoproliferative disorder. Examples of lymphoproliferative disorders include leukemia, follicular lymphoma, chronic lymphocytic leukemia, acute lymphoblastic leukemia, hairy cell leukemia, B-cell lymphoma, T-cell lymphomas, multiple myeloma, Waldenstrom's macroglobulinemia, Wiskott-Aldrich syndrome, Lymphocyte-variant hypereosinophilia, post-transplant lymphoproliferative disorder, autoimmune lymphoproliferative syndrome (ALPS) or Lymphoid interstitial pneumonia.

The trimmed sequence reads do not comprise an NGS-compatible adapter sequence. The clonal V-J segment may comprise any one of the 46-52 functional or 30 non-functional variable (V) gene segments present in the human genome. Additionally or alternatively, the clonal V-J segment may comprise any one of the 6 functional joining (J) gene segments present in the human genome. Additionally or alternatively, the clonal V-J segment may further comprise any one of the 27 functional diversity (D) gene segments present in the human genome.

The term “adapter” refers to a short, chemically synthesized, nucleic acid sequence which can be used to ligate to the end of a nucleic acid sequence in order to facilitate attachment to another molecule. The adapter can be single-stranded or double-stranded. An adapter can incorporate a short (typically less than 50 base pairs) sequence useful for PCR amplification or sequencing

The terms “complementary” or “complementarity” as used herein with reference to polynucleotides (i.e., a sequence of nucleotides such as an oligonucleotide or a target nucleic acid) refer to the base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-S.” Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

“Next-generation sequencing or NGS” as used herein, refers to any sequencing method that determines the nucleotide sequence of either individual nucleic acid molecules (e.g., in single molecule sequencing) or clonally expanded proxies for individual nucleic acid molecules in a high throughput parallel fashion (e.g., greater than 103, 104, 105 or more molecules are sequenced simultaneously). In one embodiment, the relative abundance of the nucleic acid species in the library can be estimated by counting the relative number of occurrences of their cognate sequences in the data generated by the sequencing experiment. Next generation sequencing methods are known in the art. Examples of Next Generation Sequencing techniques include, but are not limited to pyrosequencing, Reversible dye-terminator sequencing, SOLiD sequencing, Ion semiconductor sequencing, Sequencing by synthesis (SBS), Helioscope single molecule sequencing etc. Next generation sequencing methods can be performed using commercially available kits and instruments from companies such as the Life Technologies/Ion Torrent PGM or Proton, the Illumina HiSEQ or MiSEQ, and the Roche/454 next generation sequencing system.

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides of the method which function as primers or probes are generally at least about 10-15 nucleotides long and more preferably at least about 15 to 35 nucleotides long, although shorter or longer oligonucleotides may be used in the method. The exact size will depend on many factors, which in turn depend on the ultimate function or use of the oligonucleotide.

As used herein, the term “primer” refers to an oligonucleotide, which is capable of acting as a point of initiation of nucleic acid sequence synthesis when placed under conditions in which synthesis of a primer extension product which is complementary to a target nucleic acid strand is induced, i.e., in the presence of different nucleotide triphosphates and a polymerase in an appropriate buffer (“buffer” includes pH, ionic strength, cofactors etc.) and at a suitable temperature. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary to the strand. The term “forward primer” as used herein means a primer that anneals to the anti-sense strand of dsDNA. A “reverse primer” anneals to the sense-strand of dsDNA.

As used herein, “primer pair” refers to a forward and reverse primer pair (i.e., a left and right primer pair) that can be used together to amplify a given region of a nucleic acid of interest.

As used herein, a “sample” refers to a substance that is being assayed for the presence of a V-J clone. Processing methods to release or otherwise make available a nucleic acid for detection are well known in the art and may include steps of nucleic acid manipulation. A biological sample may be a body fluid or a tissue sample. In some cases, a biological sample may consist of or comprise blood, plasma, sera, urine, feces, epidermal sample, vaginal sample, skin sample, cheek swab, sperm, amniotic fluid, cultured cells, bone marrow sample, tumor biopsies, aspirate and/or chorionic villi, cultured cells, and the like. Fresh, fixed or frozen tissues may also be used.

1. A computer-implemented method to identify at least one primer of assays utilized in next-generation sequencing of a sample, comprising:
generating, by a computer server including one or more processors, from genomic data received from the next generation sequencing device, a plurality of sequence reads derived from biological samples that have been processed with forward primers and reverse primers of a next generation sequencing assay;
generating, by the computer server, a plurality of V-J gene segments by performing a lookup of each sequence read in the plurality of sequence reads in a genome database;
comparing by the computer server, each V-J gene segment of the plurality of V-J gene segments with the genomic data received from the next generation sequencing device to identify for the corresponding V-J gene segment a first number of nucleotides located upstream of the corresponding V-J gene segment and a second number of nucleotides located downstream of the corresponding V-J gene segment;
grouping, by the computer server, the plurality of V-J gene segments into a plurality of groups, each group including V-J gene segments having a same V-J identity;
for each group of the plurality of groups:
aligning by the computer server, for the V-J gene segments within the group, respective second number of nucleotides located downstream of the V-J gene segment;
aligning by the computer server, for the V-J gene segments within the group, respective first number of nucleotides located upstream of the V-J gene segment;
determining by the computer server, for the aligned respective first number of nucleotides located upstream of the V-J gene segment, at each nucleotide position, a nucleotide identity corresponding to a consensus policy to generate a forward primer consensus sequence;
determining, by the computer server, for the aligned respective second number of nucleotides located downstream of the V-J gene segment, at each nucleotide position, a nucleotide identity corresponding to the consensus policy to generate a reverse primer consensus sequence; and
identifying by the computer server, a plurality of forward primer consensus sequences as the forward primers of the next generation sequencing assay and identifying a plurality of reverse primer consensus sequences as the reverse primers of the next generation sequencing assay, optionally wherein at least one or more of the plurality of V-J gene segments further comprise a Diversity (D) region.

2. (canceled)

3. The method of claim 1, wherein the biological sample comprises nucleic acids selected from the group consisting of DNA and RNA, optionally wherein the nucleic acids are derived from one or more of CD4+ helper T cells, CD8+ cytotoxic T cells, memory T cells, gamma-delta T cells, regulatory T cells, plasma cells, memory B cells, follicular B cells, marginal zone B cells, or regulatory B cells.

4. (canceled)

5. (canceled)

6. (canceled)

7. (canceled)

8. The method of claim 1, wherein the assays utilized in next-generation sequencing of the sample are selected from the group consisting of IGH FR1 assay, IGH FR2 assay, IGH FR3 assay, IGHV leader somatic hypermutation assay, TRG assay, and IGK assay.

9. The method of claim 1, wherein the reverse primers or forward primers are between 20-30 base pairs in length, optionally wherein the reverse primers and the forward primers further comprise a NGS-compatible adapter sequence.

10. (canceled)

11. (canceled)

12. (canceled)

13. (canceled)

14. The method of claim 1, wherein comparing each V-J gene segment of the plurality of V-J gene segments with the genomic data received from the next generation sequencing device includes comparing by the computer server, each V-J gene segment of the plurality of V-J gene segments to the plurality of sequence reads derived from biological samples.

15. The method of claim 1, comprising:
accessing, by the computer server over a communication channel, the genome database to perform the lookup of each sequence read in the plurality of sequence reads in the genome database.

16. The method of claim 1, comprising:
storing, by the computer server in a first array data structure in memory, the first number of nucleotides located upstream of the V-J gene segment, one dimension of the first array data structure being indexed to a position of a nucleotide;
determining, by the computer server at each position along the one dimension of the first array data structure, the nucleotide identity corresponding to the consensus policy; and
generating, by the computer server, the forward primer consensus sequence based on the nucleotide identities determined for at least two positions along the one dimension of the first array data structure.

17. The method of claim 1, comprising:
storing, by the computer server in a second array data structure in memory, the second number of nucleotides located downstream of the V-J gene segment, one dimension of the second array data structure being indexed to a position of a nucleotide;
determining, by the computer server at each position along the one dimension of the second array data structure, the nucleotide identity corresponding to the consensus policy; and
generating, by the computer server, the reverse primer consensus sequence based on the nucleotide identities determined for at least two positions along the one dimension of the second array data structure.

18. A system comprising:
one or more processors;
a memory coupled to the one or more processors, the memory storing computer-executable instructions, which when executed by the one or more processors, causes the one or more processors to:
generate, from genomic data received from the next generation sequencing device, a plurality of sequence reads derived from biological samples that have been processed with forward primers and reverse primers of a next generation sequencing assay;
generate a plurality of V-J gene segments by performing a lookup of each sequence read in the plurality of sequence reads in a genome database;
compare each V-J gene segment of the plurality of V-J gene segments with the genomic data received from the next generation sequencing device to identify for the corresponding V-J gene segment a first number of nucleotides located upstream of the corresponding V-J gene segment and a second number of nucleotides located downstream of the corresponding V-J gene segment;
group the plurality of V-J gene segments into a plurality of groups, each group including V-J gene segments having a same V-J identity;
for each group of the plurality of groups:
align, for the V-J gene segments within the group, respective second number of nucleotides located downstream of the V-J gene segment;
align, for the V-J gene segments within the group, respective first number of nucleotides located upstream of the V-J gene segment;
determine, for the aligned respective first number of nucleotides located upstream of the V-J gene segment, at each nucleotide position, a nucleotide identity corresponding to a consensus policy to generate a forward primer consensus sequence;
determine, for the aligned respective second number of nucleotides located downstream of the V-J gene segment, at each nucleotide position, a nucleotide identity corresponding to the consensus policy to generate a reverse primer consensus sequence; and
identify a plurality of forward primer consensus sequences as the forward primers of the next generation sequencing assay and identifying a plurality of reverse primer consensus sequences as the reverse primers of the next generation sequencing assay, optionally wherein at least one or more of the plurality of V-J gene segments further comprise a Diversity (D) region.

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. A computer readable storage medium storing processor-executable instructions which, when executed by the at least one processor, causes the at least one processor to:
generate, from genomic data received from the next generation sequencing device, a plurality of sequence reads derived from biological samples that have been processed with forward primers and reverse primers of a next generation sequencing assay;
generate a plurality of V-J gene segments by performing a lookup of each sequence read in the plurality of sequence reads in a genome database;
compare each V-J gene segment of the plurality of V-J gene segments with the genomic data received from the next generation sequencing device to identify for the corresponding V-J gene segment a first number of nucleotides located upstream of the corresponding V-J gene segment and a second number of nucleotides located downstream of the corresponding V-J gene segment;
group the plurality of V-J gene segments into a plurality of groups, each group including V-J gene segments having a same V-J identity;
for each group of the plurality of groups:
align, for the V-J gene segments within the group, respective second number of nucleotides located downstream of the V-J gene segment;
align, for the V-J gene segments within the group, respective first number of nucleotides located upstream of the V-J gene segment;
determine, for the aligned respective first number of nucleotides located upstream of the V-J gene segment, at each nucleotide position, a nucleotide identity corresponding to a consensus policy to generate a forward primer consensus sequence;
determine, for the aligned respective second number of nucleotides located downstream of the V-J gene segment, at each nucleotide position, a nucleotide identity corresponding to the consensus policy to generate a reverse primer consensus sequence; and
identify a plurality of forward primer consensus sequences as the forward primers of the next generation sequencing assay and identifying a plurality of reverse primer consensus sequences as the reverse primers of the next generation sequencing assay, optionally wherein at least one or more of the plurality of V-J gene segments further comprise a Diversity (D) region.

36. (canceled)

37. (canceled)

21. (canceled)

38. (canceled)

39. (canceled)

40. (canceled)

41. (canceled)

42. (canceled)

43. (canceled)

44. (canceled)

45. (canceled)

46. (canceled)

47. The computer readable storage medium of claim 35, wherein comparing each V-J gene segment of the plurality of V-J gene segments with the genomic data received from the next generation sequencing device includes comparing by the computer server, each V-J gene segment of the plurality of V-J gene segments to the plurality of sequence reads derived from biological samples.

48. The computer readable storage medium of claim 35, the instructions causing the one or more processors to:
access, by the computer server over a communication channel, the genome database to perform the lookup of each sequence read in the plurality of sequence reads in the genome database.

49. The computer readable storage medium of claim 35, the instructions causing the one or more processors to:
store, by the computer server in a first array data structure in memory, the first number of nucleotides located upstream of the V-J gene segment, one dimension of the first array data structure being indexed to a position of a nucleotide;
determine, by the computer server at each position along the one dimension of the first array data structure, the nucleotide identity corresponding to the consensus policy; and
generate, by the computer server, the forward primer consensus sequence based on the nucleotide identities determined for at least two positions along the one dimension of the first array data structure.

50. The computer readable storage medium of claim 35, the instructions causing the one or more processors to:
store, by the computer server in a second array data structure in memory, the second number of nucleotides located downstream of the V-J gene segment, one dimension of the second array data structure being indexed to a position of a nucleotide;
determine, by the computer server at each position along the one dimension of the second array data structure, the nucleotide identity corresponding to the consensus policy; and
generate, by the computer server, the reverse primer consensus sequence based on the nucleotide identities determined for at least two positions along the one dimension of the second array data structure.

51. A computer-implemented method for detecting at least one clonal V-J gene segment in biological samples obtained from subjects, comprising:
receiving, by a computer server including one or more processors, from a next generation sequencing device, a plurality of sequence reads associated with a sample obtained from a subject, each sequence read representing at least one of coding gene segments or non-coding gene segments;
removing, by the computer server, for each sequence read of the plurality of sequence reads, a respective forward primer sequence and a respective reverse primer sequence to generate a corresponding trimmed sequence read;
identifying, by the computer server, from trimmed sequence reads generated from the plurality of sequence reads, a plurality of groups of trimmed sequence reads, each group including trimmed sequence reads having a same sequence identity;
selecting, by the computer server, one trimmed sequence read from each of the plurality of groups to form a selected set of trimmed sequence reads;
determining, by the computer server, for each trimmed sequence read in the selected set of trimmed sequence reads, a V-J identity by comparing the trimmed sequence read to a human genome database that includes associations between nucleotide sequences and V-J identities;
determining, by the computer server, for each V-J identity corresponding to a group of the plurality of groups of trimmed sequence reads, a respective frequency of the V-J identity based on a number of trimmed sequence reads included in the group;
identifying, by the computer server, based on the respective frequency of the V-J identity corresponding to a first group of the plurality of groups of trimmed sequence reads, at least one clone of the V-J identity based on a clonal detection policy optionally wherein the at least one clonal V-J gene segment further comprises a Diversity (D) region.

52. (canceled)

53. The method of claim 51, wherein the biological samples comprise nucleic acids selected from the group consisting of DNA and RNA, optionally wherein the nucleic acids are derived from one or more of CD4+ helper T cells, CD8+ cytotoxic T cells, memory T cells, gamma-delta T cells, regulatory T cells, plasma cells, memory B cells, follicular B cells, marginal zone B cells, or regulatory B cells.

54. (canceled)

55. (canceled)

56. (canceled)

57. (canceled)

58. (canceled)

59. (canceled)

60. (canceled)

61. (canceled)

62. (canceled)

63. A system comprising:
one or more processors;
a memory coupled to the one or more processors, the memory storing computer-executable instructions, which when executed by the one or more processors, causes the one or more processors to:
receive, by a computer server including one or more processors, from a next generation sequencing device, a plurality of sequence reads associated with a sample obtained from a subject, each sequence read representing at least one of coding gene segments or non-coding gene segments;
remove, by the computer server, for each sequence read of the plurality of sequence reads, a respective forward primer sequence and a respective reverse primer sequence to generate a corresponding trimmed sequence read;
identify, by the computer server, from trimmed sequence reads generated from the plurality of sequence reads, a plurality of groups of trimmed sequence reads, each group including trimmed sequence reads having a same sequence identity;
select, by the computer server, one trimmed sequence read from each of the plurality of groups to form a selected set of trimmed sequence reads;
determine, by the computer server, for each trimmed sequence read in the selected set of trimmed sequence reads, a V-J identity by comparing the trimmed sequence read to a human genome database that includes associations between nucleotide sequences and V-J identities;
determine, by the computer server, for each V-J identity corresponding to a group of the plurality of groups of trimmed sequence reads, a respective frequency of the V-J identity based on a number of trimmed sequence reads included in the group;
identify, by the computer server, based on the respective frequency of the V-J identity corresponding to a first group of the plurality of groups of trimmed sequence reads, at least one clone of the V-J identity based on a clonal detection policy, optionally wherein the at least one clonal V-J gene segment further comprise a Diversity (D) region.

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. A computer readable storage medium storing processor-executable instructions which, when executed by the at least one processor, causes the at least one processor to:
receive, by a computer server including one or more processors, from a next generation sequencing device, a plurality of sequence reads associated with a sample obtained from a subject, each sequence read representing at least one of coding gene segments or non-coding gene segments;
remove, by the computer server, for each sequence read of the plurality of sequence reads, a respective forward primer sequence and a respective reverse primer sequence to generate a corresponding trimmed sequence read;
identify, by the computer server, from trimmed sequence reads generated from the plurality of sequence reads, a plurality of groups of trimmed sequence reads, each group including trimmed sequence reads having a same sequence identity;
select, by the computer server, one trimmed sequence read from each of the plurality of groups to form a selected set of trimmed sequence reads;
determine, by the computer server, for each trimmed sequence read in the selected set of trimmed sequence reads, a V-J identity by comparing the trimmed sequence read to a human genome database that includes associations between nucleotide sequences and V-J identities;
determine, by the computer server, for each V-J identity corresponding to a group of the plurality of groups of trimmed sequence reads, a respective frequency of the V-J identity based on a number of trimmed sequence reads included in the group;
identify, by the computer server, based on the respective frequency of the V-J identity corresponding to a first group of the plurality of groups of trimmed sequence reads, at least one clone of the V-J identity based on a clonal detection policy.

76. The computer readable storage medium of claim 75, wherein the at least one clonal V-J gene segment further comprise a Diversity (D) region.

77. The computer readable storage medium of claim 75, wherein the biological samples comprise nucleic acids selected from the group consisting of DNA and RNA, optionally wherein the nucleic acids are derived from CD4+ helper T cells, CD8+ cytotoxic T cells, memory T cells, gamma-delta T cells, regulatory T cells, plasma cells, memory B cells, follicular B cells, marginal zone B cells, or regulatory B cells.

78. (canceled)

79. (canceled)

80. (canceled)

81. (canceled)

82. (canceled)

83. (canceled)

84. (canceled)

85. (canceled)

86. (canceled)
